(19) United States
(12) Patent Application Publication
(10) Pub. No.: US 2025/0060362 A1
(43) Pub. Date: Feb. 20, 2025

(54) EXCIPLEX-BASED METHODS FOR CONTAMINANT AND DISEASE DETECTION AND METHODS FOR USE OF EXTRACTION BUFFERS

(71) Applicant: Daryl Staveness, Ann Arbor, MI (US)

(72) Inventor: Daryl Staveness, Ann Arbor, MI (US)

(21) Appl. No.: 18/716,297

(22) PCT Filed: Dec. 6, 2022
(86) PCT No.: PCT/US22/52041

§ 371 Date: Jun. 4, 2024

Related U.S. Application Data
(60) Provisional application No. 63/286,375, filed on Dec. 6, 2021.

(51) Int. Cl. G01N 33/53; G01N 33/02

(52) U.S. Cl. CPC G01N 33/5308; G01N 33/02

(57) ABSTRACT

Disclosed are methods of generating spectroscopic profiles for detecting animal and human food contaminants and diseases, wherein the invention relates to methods and devices for using unique detector compounds to generate and detect exciplexes by including the use of unique extraction buffers for solubilization of analytes from mixtures for subsequent analysis with immunoassay technologies.

### BACKGROUND OF THE INVENTION

### 1. Field of the Invention

The present invention relates to a method of generating spectroscopic profiles for detecting animal and human food contaminants and diseases, and more particularly, the invention relates to methods and devices for using unique detector compounds to generate and detect exciplexes. Various aspects of the present invention especially relates to methods including using extraction buffers, and more particularly, the invention relates to methods for using unique extraction buffers for solubilization of analytes from mixtures for subsequent analysis with immunoassay technologies.

### 2. Description of the Prior Art

Conventional detection of fungi, mold and other contaminants in animal feed and human food has been attempted using varying methods, including liquid extraction, electrochemical, hydrogel, and polymeric methods to solve the problem of detecting various contaminants and diseases, such as mycotoxins including aflatoxins, zearalenones, ochratoxin A and/or deoxynivalenol (DON), among others. Prior attempts to detect such contaminants have included ultraviolet, fluorescent and visual methods.

With a measure of success, prior attempts to detect such contaminants have included ultraviolet, fluorescent and visual methods. New methods are needed in the industry to provide more efficient methods for detection of a larger number of contaminants.

Therefore, it is a desire of the mycotoxin detection industry to provide an on-site contaminant detection method capable of working across many types and forms of animal, fish and plant feeds.

The use of a novel extraction buffer composition technology is disclosed for analyzing certain analytes, including mycotoxins in cereal grains. Again, previously, one must note that conventional detection of fungi, mold and other contaminants in animal feed and human food has been attempted using varying traditional methods, such as liquid extraction, electrochemical, hydrogel, and polymeric methods to solve the problem of detecting various contaminants and diseases, such as mycotoxins including aflatoxins, zearalenones, ochratoxin A and/or deoxynivalenol (DON), among others. With a measure of success, prior attempts to detect such contaminants have included ultraviolet, fluorescent and visual methods. New methods are needed in the industry to provide more efficient and versatile methods for detection of a larger number of contaminants in a larger number of feeds, foods, and ingredients.

### SUMMARY OF THE INVENTION

In accordance with the above-noted advantages and desires of the industry, the present invention provides a new and superior method and device of generating widespread spectroscopic detectable signals by the addition of detector compounds and optional buffers to samples of contaminated animal and human feed, as well as providing a capability to detect human and animal diseases by detecting metabolites of said diseases. Furthermore, crop protection may be facilitated with predictive metrics for disease onset, especially in crops including, but not limited to, feeds such as cereal grains, strawberries, and vegetables among others. This overcomes many of the aforementioned problems with the prior art because the instant method is versatile enough to work across many feeds. Furthermore, it enables high extraction efficiencies as well as providing operationally simple spectroscopic assays. Previous methods were inadequate because they included multiple step methods that were time consuming and bothersome in comparison to the present invention.

One specific preferred aspect has certain features including a method of mixing a buffer and a detector compound with a sample food stock that is suspected of being contaminated with an analyte. Although this method will be described in greater detail below, the general steps include mixing a suspected contaminated food sample with a buffer and detector compound solution for less than 2 minutes, filtering the resulting slurry, followed by taking an aliquot of the filtrate and subjecting it to light emission sources for spectroscopic detection, including evaluations on a portable spectrophotometer/fluorimeter.

In another aspect, there is disclosed a novel diagnostic technology for detecting mycotoxins in animal feed. Photochemical detection assays are necessary to quantify agriculturally-relevant mycotoxins within crude feed extracts, which will subsequently be paired with on-site detection devices, generating on-site, for example at the mill or on the farm, mycotoxin quantification capabilities. The present invention is a novel photochemical method for detecting mycotoxins in human and animal feeds which deviates from traditional photometric or colorimetric assays such as immunofluorescence assays, by directly manipulating the photophysical properties of the toxin itself. This technique creates toxin-specific signals by selectively complexing the toxin of interest with a preselected detector compund and thus generating a new species with unique and quantifiable spectroscopic profiles. In the ground state, this would qualify as an electron donor-acceptor complex (EDA complex), and in the excited state, this is defined as an exciplex. Both states are part of a single continuum of reactivity and photochemical behavior, including the quantifiable spectroscopic outputs that are the subject of this invention. This is a fundamentally novel mechanism for identifying and quantifying small molecule analytes. Significantly, the assay concept is designed to address key commercial pain points. Such a photochemical strategy minimizes user-based manipulations, enabling fast and operationally-simple assay protocols that are operative in even highly complex feed samples. Furthermore, and beyond mycotoxins, this method will also build the foundation for a new paradigm in diagnostic science, unveiling a multitude of downstream applications for the broader scientific community.

The present invention is particularly useful for on-farm contaminant detection industries that require efficient methods and devices for broad based evaluations. The applications are too numerous to mention here.

Certain features include a method of mixing a buffer and a detector compound with a sample feed/food stock that is suspected of being contaminated with an analyte. In this aspect, there are disclosed novel diagnostic technologies for detecting various analytes, including mycotoxins. Photochemical detection assays are necessary to quantify agriculturally-relevant analytes within various sources including crude feed extracts, which will subsequently be paired with on-site detection devices, generating on-site analyte quantification capabilities. This is a novel photochemical method for detecting analytes in various sources, including animal feed, which deviates from traditional photometric assays (e.g. immunofluorescence assays) by directly manipulating the photophysical properties of the toxin itself. In one of the aspects, these techniques create toxin-specific signals by selectively complexing the toxin of interest and thus generating a new species with unique and quantifiable spectroscopic profiles. In the ground state, this is would qualify as an electron donor-acceptor complex (EDA complex), and in the excited state, this is defined as an exciplex; both states are part of a single continuum of reactivity and photochemical behavior, including the quantifiable spectroscopic outputs that are the subject of this invention. This is a fundamentally novel mechanism for identifying and quantifying small molecule analytes, possible only through the molecular-level design. Significantly, the assay concept is designed to address key commercial pain points. This photochemical strategy minimizes user-based manipulations, enabling fast and operationally-simple assay protocols that are operative in even highly complex feed samples. Furthermore, and beyond mycotoxins, this method will also build the foundation for a new paradigm in diagnostic science, unveiling a multitude of downstream applications for the broader scientific community.

The speed and simplicity of these improvements with the instant photochemical detection method provides a competitive advantage in the market. Delivering the versatility to move on-farm will directly enable proactive mycotoxin remediation strategies. Promoting informed implementation of remediation products not only increases feed additive sales for animal nutrition firms but also improves the productivity and profitability for livestock farmers. More importantly, the collective effect is a more efficient and productive food chain, providing a much-needed contribution to food security and sustainability.

Although the invention will be described by way of examples herein, see below for specific aspects having certain features, it must also be realized that minor modifications that do not require undo experimentation on the part of the practitioner are covered within the scope and breadth of this invention. Additional advantages and other novel features of the present invention will be set forth in the description that follows and in particular will be apparent to those skilled in the art upon examination or may be learned within the practice of the invention. Therefore, the invention is capable of many other different aspects and its details are capable of modifications of various aspects which will be obvious to those of ordinary skill in the art all without departing from the spirit of the present invention. Accordingly, the rest of the description will be regarded as illustrative rather than restrictive.

### BRIEF DESCRIPTION OF THE DRAWINGS

For a further understanding of the nature and advantages of the expected scope and various aspects of the present invention, reference shall be made to the following detailed description, and when taken in conjunction with the accompanying drawings, in which like parts are given the same reference numerals, and wherein:

FIG. 1 is a flow chart diagram showing steps in accordance with the present invention;

FIG. 2 is a graph of emission intensity with excitation;

FIG. 3A is a graph of emission data for aflatoxin selectivity;

FIG. 3B is a graph of emission data for aflatoxin selectivity for ochratoxin selectivity;

FIG. 4A is a graph of absorbance data;

FIG. 4B is a graph of emission data;

FIG. 5A is a graph of absorbance data; for zearalenone;

FIG. 5B is a graph of emission data; for zearalenone;

FIG. 6A is a graph of emission data at 390 nm;

FIG. 6B is a graph of emission data at 450 nm;

FIG. 7 is a blackbox depiction of a spectrometer for detecting emissions;

FIG. 8A is a graph of signal output vs. wavelength for absorbance data with detector compound background subtracted, normalized to toxin; and

FIG. 8B is a graph of absorbance vs. wavelength for absorbance data with detector compound background subtracted, normalized.

For a further understanding of the nature and advantages of the expected scope and various aspects of the present invention, reference shall be made to the following detailed description, and when taken in conjunction with the accompanying drawings, in which like parts are given the same reference numerals.

### DETAILED DESCRIPTION OF THE INVENTION

In accordance with the present invention, disclosed is a novel method and compounds for detecting contaminants in human and animal feeds, along with detection and quantification of numerous other purposes. These exciplex-based detection strategies find superior utility with wide spectrum detection of mycotoxin contamination in various feeds.

The present technology is designed to generate unique and quantifiable spectroscopic profiles for an analyte of interest through the use of buffer components and “detector compounds.” The function of the detector compound is to form a non-covalent association with an analyte of interest forming a non-covalent complex, manipulating the ground state properties to generate more readily-accessible electronic transitions. Upon irradiation with a specifically chosen wavelength, the non-covalent complex will be promoted to its excited state, where it is defined as an exciplex. This exciplex is a branch point for multiple different modes for light emission, including: 1) direct emission from a singlet state exciplex; 2) dissociation to the individual species, one of which is necessarily in the singlet excited state, followed by emission from its excited state species; 3) intersystem crossing to a triplet state exciplex followed by emission; 4) formation of ternary or higher order complexes that are emissive; 5) exchange processes, either stepwise or concerted, with another species in solution that can generate a new exciplex; 6) energy transfer to a secondary component in solution, and emission from the newly excited species; 7) electron transfer to form radical anion/cation species to produce a more readily fluorescent/phosphorescent species; and 8) any combination of the above.

It is also possible that the true mechanism does not have any relation to the non-covalent associations, for example energy transfer from a photosensitizer detector compound; photochemistry of covalent conjugates; and toxin-based quenching of alternative photometric mechanisms. Regardless of the mechanism, the function of the detector compound is defined as the manipulation of the emissive properties of the system in the presence of the analyte of interest in a quantifiable and reproducible manner. This provision is designed to protect a specific set of conditions that generate the function of spectroscopy-based identification and quantification.

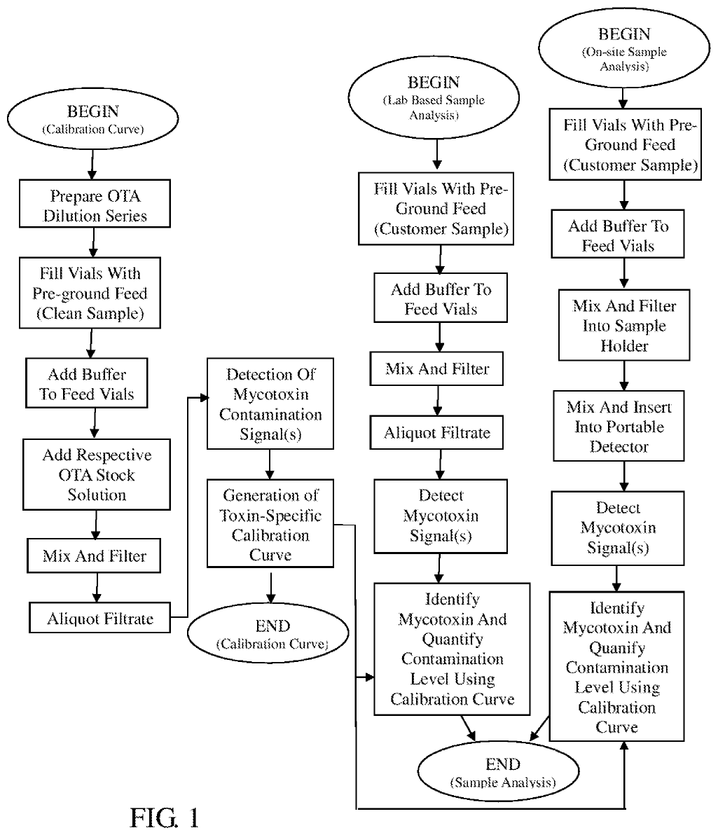

Looking now to FIG. 1, shown is a flow chart diagram illustrating a representative series of steps in accordance with the present invention, for both detection and quantification of contaminants, including mycotoxins. Essentially, pre-ground feed that is suspected of being contaminated is mixed with detector compounds and/or buffer materials before being subjected to a light source which will indicate presence of contaminants and how much of them are present.

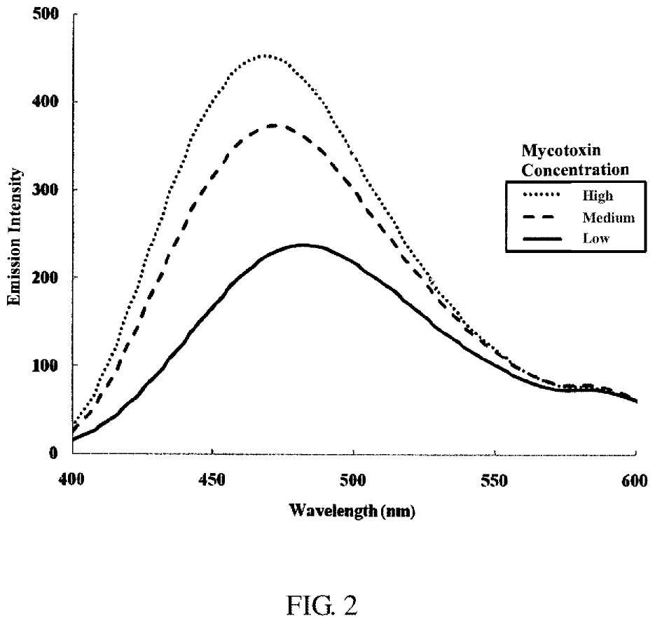

FIG. 2 is a graph of emissivity versus wavelength resulting from experiments with the best mode of the products.

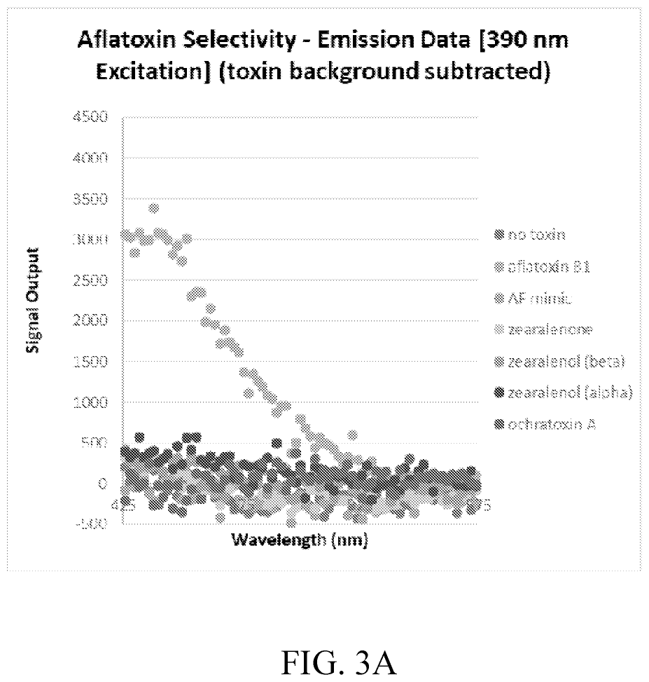

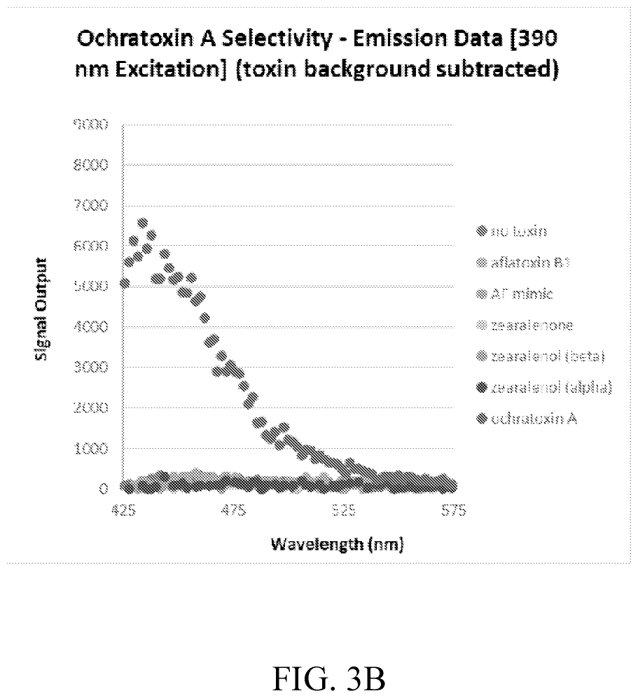

FIGS. 3A and 3B are a set of graphs showing the emission data from samples containing various mycotoxins. In these instances, the presence of the detector compound effects signal generation for only a single mycotoxin in the panel, aflatoxin B1 in one case and ochratoxin A in the second. This data is representative of the ability of this invention to employ a given detector compound to controllably generate quantitative signal for a given mycotoxin of interest.

The buffer components and detector compounds employed for these representative data sets are as follows: 1) AFB1-selective performance: 250 mM S01, pH 6, 50 mM S70, 50 mM H20, 50 mM P01 [avg. m.w.=6000] containing 10 uM E04; 2) OTA-selective performance: 100 mM S01, pH 6, 50 mM P02 [avg. m.w.=2000] containing 10 uM H08. This analysis was performed on a sample of chicken scratch (a 5-grain blend comprised of corn, millet, barley, wheat, and sunflower seed) in the manner described herein.

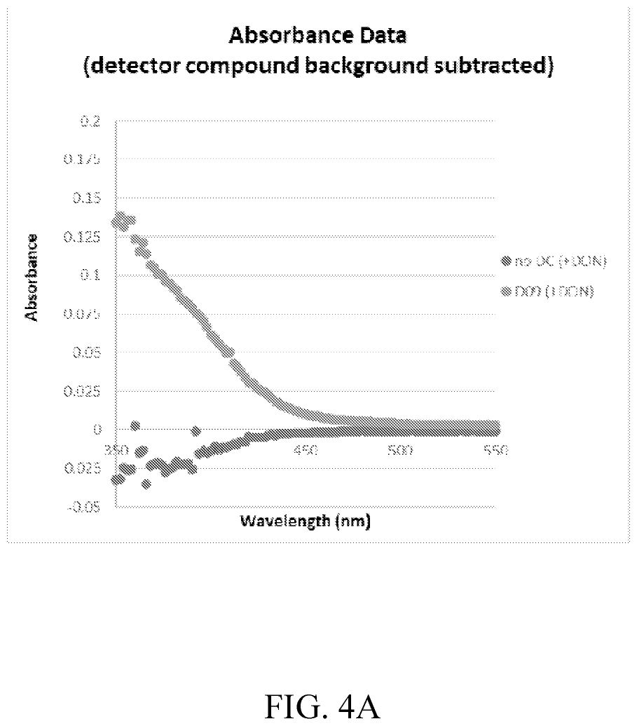

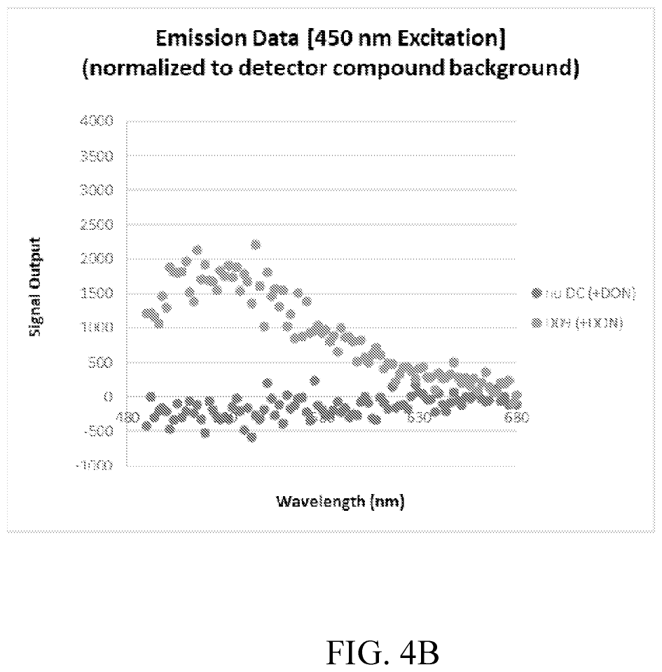

FIG. 4 is a set of graphs detailing the absorbance data and emission data, respectively, from samples containing a mycotoxin, namely, deoxynivalenol. The increased absorbance in the presence of detector compound and the corresponding increased emission upon irradiation is representative of the ability of this invention to leverage ground-state manipulations for the generation of quantifiable emissive signals. The buffer components and detector compound employed for these representative data sets are as follows: 125 mM S51, pH 8.5, 125 mM R90, 16.66 mM P01 [avg. m.w.=6000] containing 10 uM D09. This analysis was performed on a sample of chicken scratch in the manner described herein.

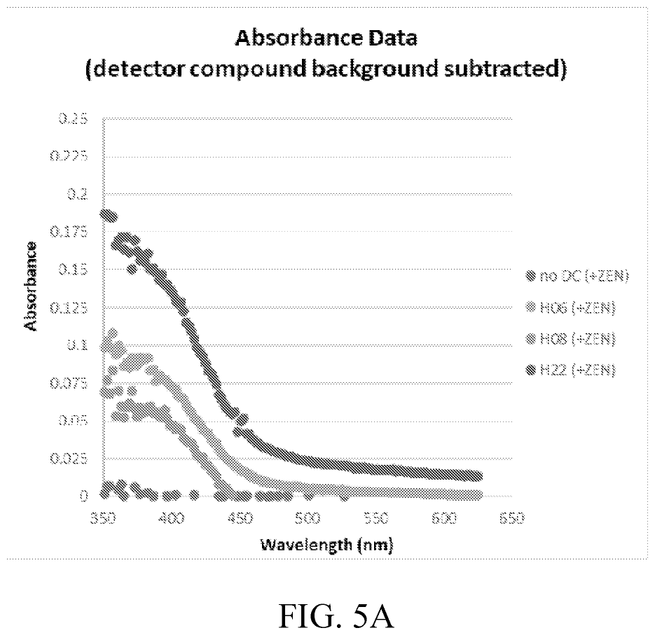

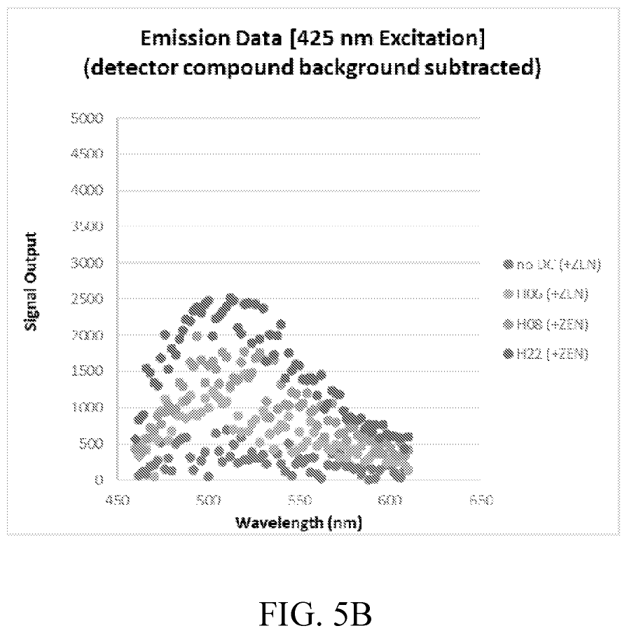

FIGS. 5A and 5B are collectively a set of graphs detailing the absorbance data and emission data, respectively, from samples containing a mycotoxin, namely, zearalenone. As above, the increased absorbance in the presence of detector compound and the corresponding increased emission upon irradiation is representative of the ability of this invention to leverage ground-state manipulations for the generation of quantifiable emissive signals. The buffer components and detector compounds employed for these representative data sets are as follows: 250 mM S41, pH 10, 25 mM F05, 50 mM P02 [avg. m.w.=2000] containing 10 μM H06 or H08 or H22. This analysis was performed on a sample of chicken scratch in the manner described herein.

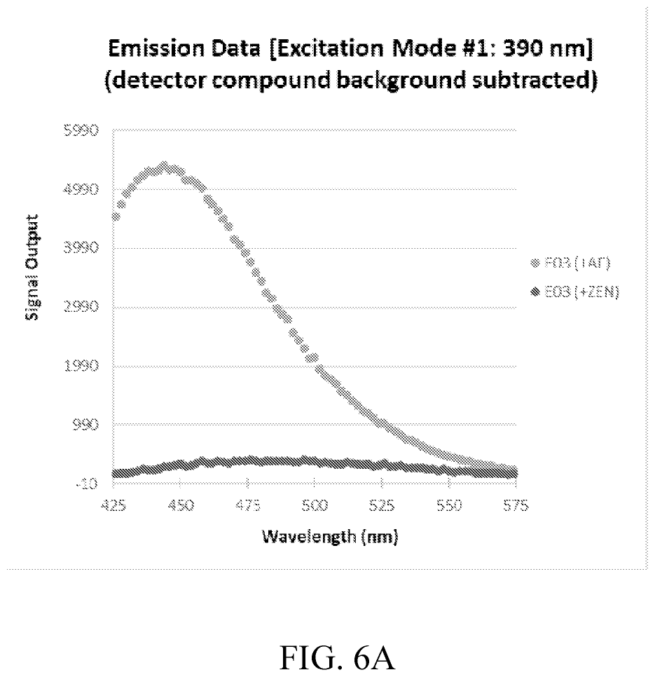

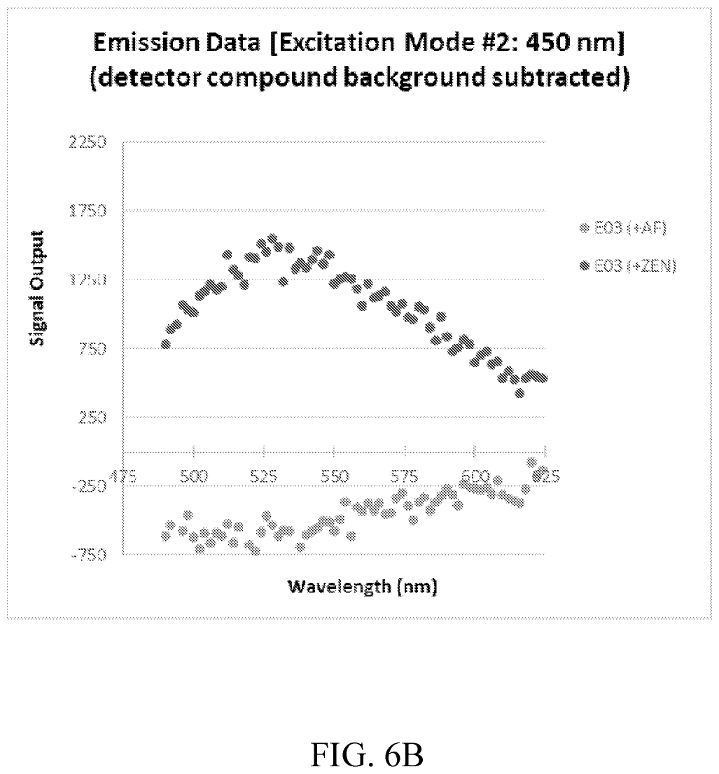

FIGS. 6A and 6B are collectively a set of graphs detailing the emission data from two separate excitation modes as applied to separate samples containing two mycotoxins, namely aflatoxin and zearalenone. In this instance, the 390 nm excitation mode led to signal enhancement for aflatoxin without generating significant response from zearalenone, while the 450 nm excitation mode led to signal enhancement for zearalenone without generating significant response from aflatoxin. This data represents the potential for this invention to employ a single formulation of extraction buffer and detector compound to create quantifiable signal for multiple mycotoxins, with the signal specificity controlled through the method of irradiation. The buffer components and detector compounds employed for these representative data sets are as follows: 100 mM S01, pH 6, 50 mM P02 [avg. m.w.=2000] with 20% DMSO containing 10 μM E03. This analysis was performed on a sample of chicken scratch in the manner described herein.

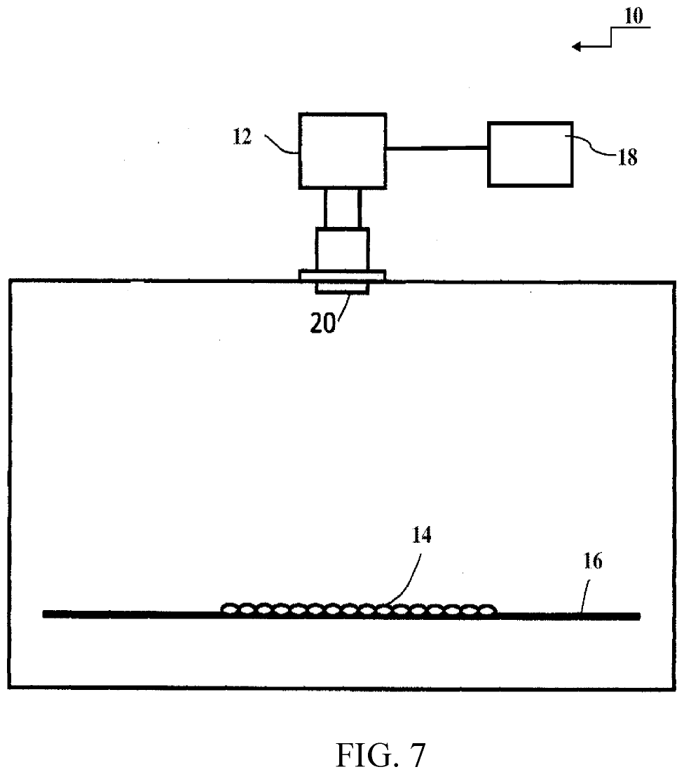

FIG. 7 is a blackbox depiction of a spectrometer for detecting emissions of the exciplex created by the addition of the abovementioned detector/buffer compounds when added to contaminated feed. The spectrometer is generally indicated by the numeral 10 and includes a light source 12 shining downwardly through lens 20 onto pre-treated sample 14 on plate 16. Light source 12 also receives the spectral image and transmits the spectral data to data receiver 18 for further analysis and recording.

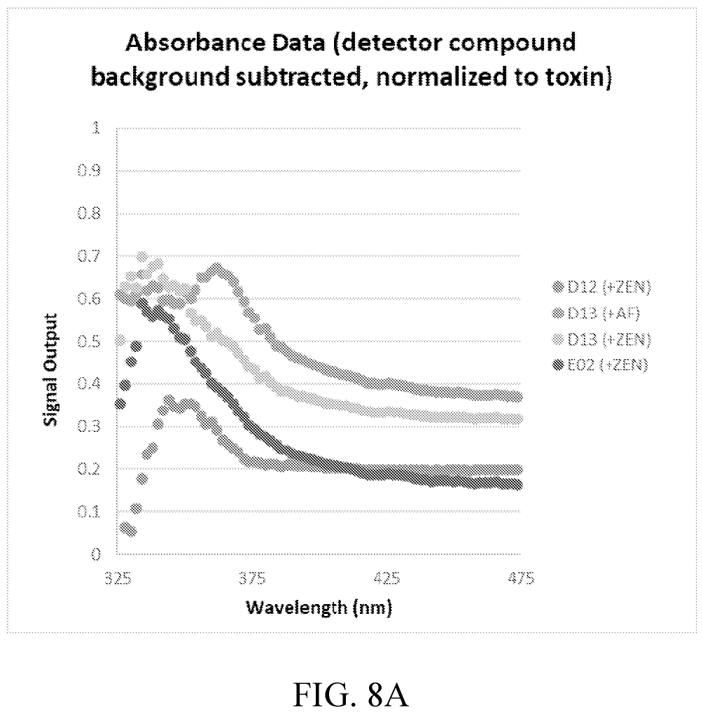

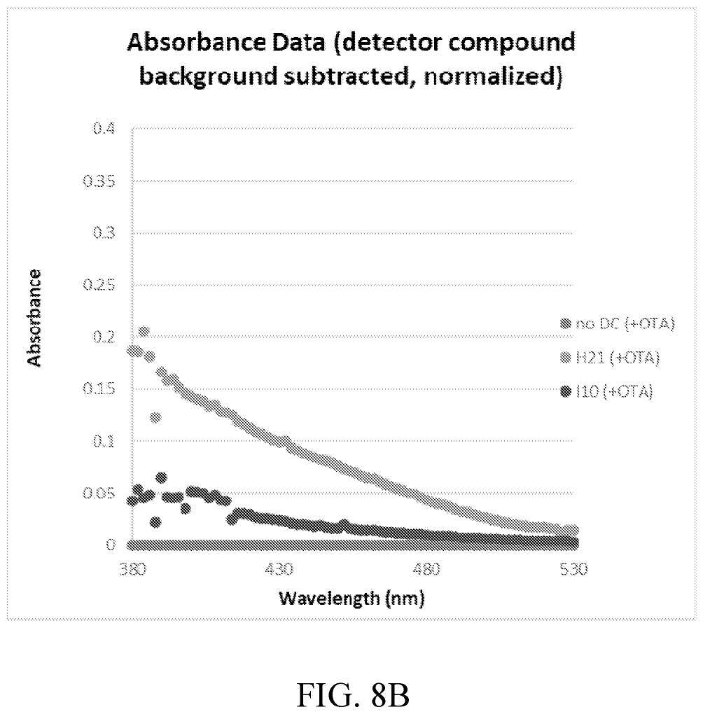

FIGS. 8A and 8B collectively show comparative graphs of signal output vs. wavelength for absorbance data with detector compound background subtracted, normalized to toxin in FIG. 8A and a graph of absorbance vs. wavelength for absorbance data with detector compound background subtracted, normalized in FIG. 8B respectively.

### EXAMPLES

Assay prototypes have been prepared and employed to generate spectroscopic data outputs and/or quantitative data for mycotoxin contamination in a variety of feeds and feed ingredients. Both naturally-contaminated and exogenously-contaminated samples are assessed to provide preliminary assessment of the present diagnostic concept.

Execution of the following examples employs one or more of the following methods, methods that are demonstrative of and consistent with the invention described herein. These methods are representative of the scope of work available through the use of this invention, but are not an exhaustive of list of possible use cases.

### General Method A—Buffer Preparation:

The principal buffer component (i.e. buffer in the highest concentration) at the amount specified is dissolved in deionized water (80% of final target volume). All additional reagents are added sequentially in the amounts specified, with the exception of digestion enzymes and co-solvents. Using a standard pH meter, the pH of the solution is adjusted to the desired final pH using either acid or base as needed (typical acid: 5 M hydrochloric acid in water; typical base: potassium hydroxide). Where applicable, digestion enzymes and/or co-solvents are added. Solution is diluted with water and mixed thoroughly to achieve the final target volume.

### General Method B—Buffer Preparation for Buffer Screening Purposes:

To prepare a series of buffer formulations with one core buffer, the Core Buffer is prepared as in General Method A with all reagents added at 1.25 times the target concentration in the final buffer formulations; digestion enzymes and/or co-solvents are not included in the Core Buffer. Core Buffer (10 mL) is aliquoted into the desired number of tubes. Additives of interest are added to the respective tubes in an amount that will deliver the target concentration at a final volume of 12.5 mL [volumes can be scaled if necessary]. The pH is measured, and if the additive has altered the pH of the Core Buffer, it is corrected at the juncture, using the same tactics as in General Method A. Digestion enzymes and/or co-solvents are added, and the solution is diluted to a final volume of 12.5 mL.

### General Method C—Detector Compound Screen:

Employing a pre-made extraction buffer, the following method represents a means of determining the influence of a detector compound candidate on a given analyte in a given medium, specifically evaluating 7 candidates against a DMSO blank [volumes and methods are readily adjusted to accommodate additional candidates, feed matrices, and/or buffers]. Prepare two 3.0 g feed samples (No Tox; +Tox) [for feeds/matrices with lower moisture content, larger samples may be necessary; all amounts should be scaled to maintain 4:1 vol: mass extraction ratio]. Add 12 mL buffer to each sample. To the No Tox sample, add 12 μL of DMSO. To the +Tox samples, add 12 μL of a stock solution of the mycotoxin (or other analyte) of interest in DMSO; stock solution must be 1,010 times the final target assay concentration. Samples are mixed by vortexing for 30 seconds [alternative mixing methods also viable; where applicable, longer mixing times are noted], before individually filtering through funnel-shaped filter papers (typical filter paper: Whatman Grade 4 filter paper, 11 cm folded into quarters). After ≤2 min of filtration, the funnel is removed, and the filtrates are individually homogenized by pipetting up and down. The filtrates are each aliquoted into 8 tubes, 495 uL each. The detector compound of interest is added to the filtrate, using 5.0 uL of a stock solution at 100 times the concentration of the final target assay concentration [typical detector compound concentration: 1.0 μM to 10 μM, as detailed in the individual examples]; each detector compound and the DMSO blank is added to two total samples, one No Tox filtrate tube and one+Tox filtrate tube. Each tube is mixed by vortexing for ˜5 seconds. Three 150 μL aliquots of each filtrate+detector compound mix are added to a 96-well plate suitable for spectroscopic measurement (either clear bottom for both Absorbance and Fluorescence measurements or black/white-bottom if only collecting Fluorescence measurements). Data is collected in a suitable platereader as per the needs of the individual trial at hand; most data that follows was collected using a Tecan M Nano platereader.

Data processing is as follows. At the wavelength of interest (either via emission data or absorbance data; wavelength of interest pre-determined through scans that identify the max Abs or Emission, either employing a single point or the average of multiple points at and around the maximum), the No Tox and +Tox trials are averaged, and standard deviations are calculated. A change in emission metric (AEm) is generated by subtracting Avg (No Tox) from Avg (+Tox) [or similarly, AA for Absorbance trials]. Signal-to-noise (S/N) is calculated from the quotient of AEm and Avg (No Tox). A statistical significance metric (SD quotient) is calculated by dividing AEm by the standard deviation of Avg (+Tox). Where applicable, a detector compound influence metric (DC Effect, expressed as a percentage) is calculated from: DC Effect=(ΔEm of detector compound trial)÷(ΔEm of DMSO blank trial)−1. Graphical representations can be created from the following, typically plotted as a function of wavelength. Toxin Background Subtracted refers to data generated from Avg (+Tox) of detector compound trial minus Avg (+Tox) of DMSO blank trial. Detector Compound Background Subtracted is equivalent to ΔEm data. Both of the preceding background subtractions can be followed by and additional normalization to the other variable (toxin or detector compound), e.g. ΔEm data of detector compound trial can be subtracted or divided by the ΔEm data of the DMSO blank trial to correct/normalize for the influence of toxin within the filtrate of interest; these calculations are denoted where applicable.

### General Method D—Buffer Background Noise Analysis:

The following method allows one to determine the ability of a given buffer formulation to reliably control the spectroscopic background noise generated by feed/food/beverage extractions. Prepare a 1 g sample of the feed matrix of interest. Add 4.0 mL of the pre-made extraction buffer of interest. Vortex for 30 seconds to mix. Filter by pouring into a funnel-shaped filter paper (typical filter paper: Whatman Grade 4 filter paper, 7 cm folded into quarters). Collect 0.5-1.5 mL of filtrate; for matrices that yield little filtrate or cloudy filtrate, each sample can be decanted into a 1.5 mL microcentrifuge tube and spun (e.g. 6000 g) in a centrifuge for 2 min, allowing the supernatant to serve the role of filtrate. Pipet three 150 μL aliquots of filtrate into 3 separate wells within a 96-well plate (or other suitable sample containment); plate must enable collection of both Absorbance and Fluorescence data. Collect data at t=15 min from the time of pouring the initial filtration; additional time points may be collected for comparison, e.g. t=75 min. Typical data collection is as follows, and may be altered to reflect the needs of a given extraction: Absorbance mode: 374 nm, 400 nm, 424 nm, and 500 nm data points are used; Fluorescence mode: for excitation wavelength=390 nm, emission data at 440 nm and 540 nm is used, for excitation wavelength 450 nm, emission data at 500 nm and 600 nm is used, for excitation wavelength=500 nm, emission data at 550 nm and 650 nm is used. Alternative combinations are also viable and can be set based on the needs of the downstream experimentation.

Data processing is as follows. At any given data point, the three trials are averaged and standard deviations are calculated. Comparative data metrics are generated by analyzing the per-cent change in Absorbance or Emission data relative to a reference buffer

### General Method E—Sample Quantification and Calibration, Spectroscopic Method:

To determine the amount of toxin in a naturally-contaminated sample, measure three 1 g samples into separate vials. Add 4.0 mL of the extraction buffer to each (detector compound introduction: detector compound may be added to buffer as a DMSO stock immediately before use or formulated along with other buffer additives as in General Method A or B). Number of sample replicates and sample size may be adjusted (ensuring that the 4:1 volume: mass ratio is main-tained); if applicable/desirable, alterations should be validated as reproducible and accurate through control studies in line with General Method C and the methods listed below. Mix by vortexing 30 sec (other mixing methods acceptable; time range may require extension for poorly-soluble analytes). Filter by pouring into three separate funnel-shaped filter papers (typical filter paper: Whatman Grade 4 filter paper, 7 cm folded into quarters). Collect 1.5-2.0 mL of filtrate; for matrices that yield little filtrate or cloudy filtrate, each sample can be decanted into a 1.5 mL microcentrifuge tube and spun (6000 g) in a centrifuge for 2 min, allowing the supernatant to serve the role of filtrate. Pipet 150 uL from each filtrate into a well within a 96-well plate (or other suitable sample containment), and measure the emission data at the wavelength of interest as produced with the requisite excitation wavelength. Data should be collected in a suitable platereader; most data that follows was collected using a Tecan M Nano platereader. Data can also be collected using other spectroscopic instruments, including portable devices; in the instance that a cuvette or test tube is necessary for sample containment, the amounts above can be scaled to generate larger volumes of filtrate (e.g. 10 g feed sample, 40 mL buffer, 10-15 mL filtrate collect, aliquot 3 mL of each filtrate into individual cuvette or test tube). Data processing is as follows. The emission data of the three trials is averaged. The background contributions are subtracted, employing pre-collected background emission data with a No Tox trial. The resulting data is the measured ΔEm, which can be converted to toxin concentration with the appropriate calibration curve (see curve generation method below). In certain instances, additional background correction processing may be necessary (e.g. feed-to-feed comparisons); in these instances, additional emission data is collected at points red-shifted≥50 nm relative to the wavelength of interest; these data points are averaged and a ratio is generated between data from the sample of interest and from the known No Tox trial. This ratio can be applied to the raw emission data prior to averaging and determining Aem.

For unit tests with samples that require exogenous introduction of analyte, the process is highly analogous to the method for naturally-contaminated samples. The exogenous introduction of toxin can be performed at two points: 1) after addition of buffer to feed, 2) after filtration. In the first instance, add 40 μL of toxin stock solution in DMSO (stock concentration=100 times the desired assay concentration, e.g. 100 nM assay concentration requires 10 μM stock concentration), then mix as prescribed. In the second instance, collect 495 μL of filtrate, add 5.0 uL of toxin stock solution, mix by briefly vortexing the tube, and aliquot as per usual. These trials are best performed in parallel with a DMSO blank trial, in which the toxin stock solution is replaced with DMSO only; this allows for more direct assessment of ΔEm. The two types of exogenous addition trials can be run in parallel; comparison of the ΔEm values can provide a metric for extraction efficiency in response to the feed matrix employed. Lastly, one can reduce the overall amount of DMSO in the assay by preparing the toxin stock solution in the buffer of interest.

To generate calibration curve data, the exogenous addition of toxin method is employed for a series of concentrations. Representative assay concentrations are: 0 nM (DMSO blank trial), 3.7 nM, 11 nM, 33 nM, 100 nM, 300 nM. The requisite series of DMSO stock solutions is prepared by first generating the highest concentration stock necessary, then executing a serial dilution (1 part stock solution to 2 parts DMSO at each tier) to complete the series. Concentrations can be adjusted to fit the needs of the curve (i.e. increasing or decreasing depending on the anticipated or desired limit of detection; expanding the range by increasing the dilution ratio). The calibration curve can be calculated from the plot of AAbs or ΔEm vs concentration. Absorbance data will yield a linear relationship. Emission data can be approximated as linear for low concentrations in the case of high sensitivity assays, but larger concentration ranges require non-linear regression methods to accurately represent the relationship. The curve of interest can then be used in the calculation of concentration in subsequent experiments (naturally-contaminated or exogenously-contaminated samples).

### General Method F—Sample Quantification and Calibration, Immunoassay Method:

To determine the amount of toxin in a naturally-contaminated sample, first measure 10 g of the feed matrix of interest into a suitable jar or tube. The following protocol is adapted from the standard protocol of Neogen's Reveal Q+ Max product line and requires certain products from said kits. Add 40 mL of extraction buffer to feed sample, and shake for 1 min [for Reveal Q+ Max protocol, a buffer solids packet is added to feed, followed by 50 mL water and a 3 min machine mix]. Separation can be achieved via filtration, as in General Method C, or via centrifugation; for the latter, decant the shaken mixture into a 1.5 mL centrifugation tube, spin for ˜2 min (any speed>2000 g will suffice), and employ the supernatant as the filtrate; syringe filtration and other methods are also viable separation techniques. Collect 400 μL of filtrate and mix with 400 μL of the supplied diluent. Transfer 400 μL of 1:1 mixture into a test strip cartridge pre-loaded with a lateral flow test strip for the toxin of interest; cartridge should be pre-loaded into Neogen's Raptor reader device (if using alternative device, follow recommended test strip loading procedure). The device supplies concentration report after incubation period, though this concentration is calculated from the calibration curve that is specific to the commercial buffer. Device software yields raw line intensity values (test line, control line) and the ratio of the two line intensities (Ratio), which can be employed for alternative calibrations. For extraction buffers other than the commercial standard, the Ratio data serves as the measured input for calculating concentration, employing the buffer-specific curve (see below).

For unit tests that require exogenous addition of the toxin/analyte, adjustments analogous to those in General Method E can be followed. For the case in which analyte is added after separation, this should be performed prior to the 1:1 dilution. As it pertains to the examples supplied herein, the additions were as follows: addition prior to mixing-50 μL analyte stock solution to 40 mL buffer after pouring into 10 g feed sample [stock solution=800 times desired filtrate concentration]; addition after separation-5.0 μL stock solution to 4.0 mL filtrate [stock solution=800 times desired filtrate concentration]. For centrifugation methods using the latter inoculation technique, multiple supernatants must be combined to generate 4.0 mL ‘filtrate’. All other steps follow the above method.

As in General Method E, calibration curve data is generated through a series of exogenous addition trials referenced to a DMSO blank trial. For the calibration curves employed herein, the concentration series was: 0 ppb [DMSO blank], 2.5 ppb, 5.0 ppb, 10 ppb, 20 ppb, 50 ppb, 75 ppb. Note that ppb, in this field, is referenced to the 10 g feed sample, thus stock solutions must be generated to recapitulate the molar concentration of the analyte in the filtrate based on the buffer: feed volume: mass ratio. The quantitative relationship employed is the logistic function generated via non-linear regression of log (Ratio) vs log (concentration).

### Example 1

Example 1 details selected examples of emission signal production from a mycotoxin, namely, ochratoxin A. In these instances, the effect of the detector compound on signal production in a given buffer is provided. The combined data represent the ability of the medium and detector compound to collectively influence the energetics of the emission signal (i.e. wavelength of maximum emission) and the magnitude, illustrating the concepts of the invention regarding detector compound-based alterations to the spectroscopic character of mycotoxins in relevant feed/food samples in a manner that can be quantified.

These examples are in tabular format, detailing selected examples of emission signal production from a mycotoxin, namely, ochratoxin A. In these instances, the effect of the detector compound on signal production in a given buffer is provided. The combined data represent the ability of the buffer formulation and detector compound to collectively influence the emission signal (wavelength of maximum emission, signal magnitude), illustrating the concepts of the invention regarding detector compound-based alterations to the spectroscopic character of mycotoxins in relevant feed/food samples in a manner that can be quantified.

| | | Detector | Avg | | | Max |
|---|---|---|---|---|---|---|
| Buffer | Feed | Compound | Signal | S/N | DC Effect | Emission |
| A | chicken scratch | H09 | 16362 | 70% | 30% | 450 |
| A | silage | H26 | 9084 | 26% | 46% | 450 |
| B | silage | D05 | 6231 | 15% | 14% | 450 |
| B | silage | D10 | 6906 | 18% | 26% | 450 |
| B | silage | D19 | 6475 | 16% | 18% | 450 |
| B | silage | E01 | 6393 | 16% | 17% | 450 |
| B | silage | E04 | 6314 | 16% | 15% | 450 |
| B | silage | H09 | 6065 | 15% | 11% | 450 |
| B | silage | H26 | 6205 | 15% | 13% | 450 |
| C | silage | D05 | 3753 | 11% | 13% | 438 |
| C | silage | D19 | 3598 | 11% | 9% | 438 |
| C | silage | E01 | 3627 | 11% | 9% | 438 |
| C | silage | E04 | 3444 | 11% | 4% | 438 |
| C | silage | H26 | 3702 | 11% | 12% | 438 |
| D | chicken scratch | H09 | 13360 | 65% | 12% | 448 |
| E | chicken scratch | D18 | 7888 | 26% | 30% | 450 |
| E | chicken scratch | E01 | 7410 | 24% | 22% | 450 |
| E | chicken scratch | E03 | 6449 | 21% | 7% | 450 |
| E | chicken scratch | E04 | 6752 | 22% | 12% | 450 |
| E | chicken scratch | E08 | 6524 | 21% | 8% | 450 |
| E | chicken scratch | H09 | 6681 | 22% | 10% | 450 |
| E | chicken scratch | H27 | 6381 | 21% | 5% | 450 |
| F | chicken scratch | D18 | 6110 | 21% | 5% | 450 |
| F | chicken scratch | H09 | 6601 | 24% | 13% | 450 |
| F | chicken scratch | H27 | 7289 | 27% | 25% | 450 |
| G | chicken scratch | D18 | 6427 | 20% | 18% | 450 |
| G | chicken scratch | E01 | 5936 | 18% | 9% | 450 |
| G | chicken scratch | E03 | 5771 | 18% | 6% | 450 |
| G | chicken scratch | E04 | 5865 | 18% | 8% | 450 |
| H | chicken scratch | D18 | 5380 | 20% | 8% | 450 |
| H | chicken scratch | E03 | 5212 | 20% | 4% | 450 |
| H | chicken scratch | E08 | 5642 | 21% | 13% | 450 |
| I | chicken scratch | H09 | 5834 | 22% | 7% | 450 |
| I | chicken scratch | H27 | 5887 | 22% | 8% | 450 |
| J | red wine | E01 | 3740 | 14% | 26% | 448 |
| J | red wine | E03 | 3036 | 19% | 2% | 448 |
| J | red wine | H09 | 3835 | 15% | 29% | 448 |
| J | red wine | H39 | 3379 | 20% | 14% | 448 |
| J | red wine | D33 | 3454 | 17% | 16% | 448 |
| K | chicken scratch | C09 | 18591 | 18% | 1% | 440 |
| K | chicken scratch | E02 | 18553 | 110% | 1% | 440 |
| K | chicken scratch | E08 | 18653 | 109% | 2% | 440 |
| K | chicken scratch | H26 | 18940 | 112% | 3% | 440 |
| K | chicken scratch | D12 | 19335 | 104% | 5% | 440 |
| K | chicken scratch | D19 | 19238 | 111% | 5% | 440 |
| L | aqua feed | EC | 4260 | 45% | 23% | 440 |

The tabular data above details the quantitative metrics collected from Fluorescence mode data collection, specifically: Avg (+Tox), S/N, DC Effect, and the wavelength of maximum emission (“Max Emission”). Excitation wavelength was 385-390 nm. The buffer formulations employed was prepared using General Method B. The buffer compositions were as follows (the respective detector compounds are listed in the table): A) 500 mM 199, pH 8 (adjusted with potassium hydroxide), 100 mM 189, 35 mM 126, 15 mM 212 [avg. m.w.=6000]; B) 500 mM 199, pH 8 (adjusted with potassium hydroxide), 100 mM 189, 35 mM 126, 0.5 mM 229, 15 mM 212 [avg. m.w.=6000]; C) 500 mM 195, pH 8 (adjusted with hydrochloric acid), 35 mM 126, 35 mM 221, 15 mM 212 [avg. m.w.=6000]; D) 500 mM 195, pH 8 (adjusted with hydrochloric acid), 100 mM 210, 35 mM 237, 35 mM 234, 15 mM 212 [avg. m.w.=6000]; E) 500 mM 199, pH 8 (adjusted with potassium hydroxide), 100 mM 189, 35 mM 126, 15 mM 212 [avg. m.w.=6000]; F) 500 mM 199, pH 8 (adjusted with potassium hydroxide), 100 mM 189, 35 mM 126, 0.5 mM 229, 15 mM 212 [avg. m.w.=6000]; G) 500 mM 199, pH 8 (adjusted with potassium hydroxide), 100 mM 189, 35 mM 12, 15 mM 212 [avg. m.w.=6000]; H) 500 mM 199, pH 8 (adjusted with potassium hydroxide), 100 mM 189, 35 mM 126, 35 mM 236, 15 mM 212 [avg. m.w.=6000]; I) 500 mM 199, pH 8 (adjusted with potassium hydroxide), 100 mM 189, 35 mM 126, 0.5 mM 229, 35 mM 236, 15 mM 212 [avg. m.w.=6000]; J) 400 mM 203, pH 7.5 (adjusted with potassium hydroxide), 100 mM 198, 35 mM 93, 35 mM 242, 45 mM 213 [avg. m.w.=2000]; K) 250 mM 190, pH 8.5 (adjusted with sodium hydroxide), 100 mM 113, 1.25 mg/mL pectinase, 15 mM 212 [avg. m.w.=6000]; L) 250 mM 190, pH 7.5 (adjusted with sodium hydroxide), 50 mM 215, 25 mM 12, 1.25 mg/mL pectinase, 35 mM 137, 350 mM 225, 15 mM 212 [avg. m.w.=6000]. The experimental protocols followed General Method C. The feed/food/beverage matrices employed were chicken scratch, corn silage, red wine, and a trout feed [Rangen; “aqua feed”], as specified; detector compounds were 57, 35, 67, 71, 73, 46, 50, 57, 74, 48, 54, 58, 63, 83, 17, 47, or 166, as specified, at 100 M; the mycotoxin employed was ochratoxin A at either 1.0 μM (A-D, K-L) or 300 nM (E-J).

The buffer components employed for these representative data sets are as follows (detector compounds are listed in the table): A) 500 mM R20, pH 8, 100 mM S40, 35 mM C03, 15 mM P01 [avg. m.w.=6000]; B) 500 mM R20, pH 8, 100 mM S40, 35 mM C03, 0.5 mM Q81, 15 mM P01 [avg. m.w.=6000]; C) 500 mM R01, pH 8, 35 mM C03, 35 mM Q40, 15 mM P01 [avg. m.w.=6000]; D) 500 mM R01, pH 8, 100 mM S03, 35 mM S52, 35 mM Q01, 15 mM P01 [avg. m.w.=6000]; E) 500 mM R20, pH 8, 100 mM S40, 35 mM C03, 15 mM P01 [avg. m.w.=6000]; F) 500 mM R20, pH 8, 100 mM S40, 35 mM C03, 0.5 mM Q81, 15 mM P01 [avg. m.w.=6000]; G) 500 mM R20, pH 8, 100 mM S40, 35 mM B10, 15 mM P01 [avg. m.w.=6000]; H) 500 mM R20, pH 8, 100 mM S40, 35 mM C03, 35 mM S51, 15 mM P01 [avg. m.w.=6000]; I) 500 mM R20, pH 8, 100 mM S40, 35 mM C03, 0.5 mM Q81, 35 mM S51, 15 mM P01 [avg. m.w.=6000]; J) 400 mM R22, pH 7.5, 100 mM Q03, 35 mM H20, 35 mM sodium borate, 45 mM P02 [avg. m.w.=2000]; K) 250 mM S41, pH 8.5, 100 mM H03, 1.25 mg/mL pectinase, 15 mM P01 [avg. m.w.=6000]; L) 250 mM S41, pH 7.5, 50 mM S70, 25 mM B10, 1.25 mg/mL pectinase, 35 mM H19, 350 mM R50, 15 mM P01 [avg. m.w.=6000]. These analyses were performed on the denoted feed/food types with either 1.0 μM OTA (A-D, K-L) or 300 nM OTA (E-J) in the manner described herein. ‘DC Effect’ is a metric generated by subtracting the emission contributions from the detector compound in the medium then normalizing to any residual native fluorescence of the toxin, thus allowing for quantification of the signal generated as a direct result of toxin and detector compound interaction (i.e. signal that is beyond the level anticipated of purely additive combination).

### Example 2

Example 2 details test line intensities of a lateral flow device immunoassay (LFD) test observed upon employing extraction buffers detailed herein. The reduction in test line intensity when using aflatoxin-contaminated corn rather than corn with no detectable amount of aflatoxin is illustrative of the capacity of these extraction buffer formulations to solubilize mycotoxins or other analytes from a commercially-relevant matrix while supporting the function of canonical immunoassay techniques for analyte quantification.

| | Normalized Test Line Intensity | |
|---|---|---|
| Buffer | no AF | AF |

| Candidate | Avg | Std Dev | Avg | Std Dev |
|---|---|---|---|---|
| Standard | 100.0 | 7% | 64.0 | 21% |
| C01 | 98.0 | 4% | 84.7 | 7% |
| C02 | 92.0 | 5% | 75.3 | 8% |
| C03 | 91.2 | 8% | 73.3 | 6% |
| C04 | 95.9 | 5% | 80.8 | 10% |
| C05 | 91.3 | 7% | 67.2 | 10% |
| C06 | 106.7 | 6% | 88.3 | 13% |
| C07 | 89.1 | 10% | 85.4 | 8% |
| C08 | 101.0 | 6% | 92.4 | 8% |
| C09 | 96.4 | 8% | 84.5 | 8% |
| C10 | 102.9 | 5% | 87.9 | 6% |

The buffers employed for these representative data sets are as follows: C01) 500 mM R01, pH 7.5, 100 mM S03, 12.5 mM P02 [avg. m.w.=2000]; C02) 500 mM R01, pH 7.5, 100 mM S03, 2.5 mM P02 [avg. m.w.=2000]; C03) 500 mM R01, pH 7.5, 100 mM S03, 0.5 mM P02 [avg. m.w.=2000]; C04) 500 mM R01, pH 7, 100 mM S03, 12.5 mM P02 [avg. m.w.=2000]; C05) 500 mM R01, pH 8, 100 mM S03, 12.5 mM P02 [avg. m.w.=2000]; C06) 500 mM R01, pH 7.5, 100 mM S03, 25 mM P02 [avg. m.w.=2000]; C07) 400 mM Q02, pH 7.5, 100 mM S03, 12.5 mM P02 [avg. m.w.=2000]; C08) 400 mM Q03, pH 7.5, 100 mM S03, 12.5 mM P02 [avg. m.w.=2000]; C09) 400 mM R20, pH 7.5, 100 mM S01, 12.5 mM P02 [avg. m.w.=2000]; C10) 400 mM R25, pH 7.5, 100 mM S03, 12.5 mM P02 [avg. m.w.=2000]. The Reveal Q+ Max from Neogen was used as a representative LFD and the standard conditions were followed except for the replacement of the standard extraction buffer with the extraction buffers from this invention.

### Example 3

FIG. 3A demonstrates toxin-selective emission signal enhancement using the exciplex-based detection technique described in this invention. Specifically, it shows the increased signal production for aflatoxin B1 in a panel of multiple mycotoxins; the other mycotoxins offer no detectable change in signal with the buffer formulation and detector compound employed in this experiment. This example illustrates the ability of this invention to controllably generate quantifiable spectroscopic signals for a specific mycotoxin of interest through the design and selec-tion of the detector compound and buffer formulation.

The data above is a graphical depiction of Fluorescence data with the toxin background subtracted. “Fluorescence” here refers to the measurement mode nomenclature as it appears in the platereader employed for this experiment (this nomenclature is consistent with many other devices of this type), in which the sample is irradiated with light of a specific excitation wavelength, and the detector reads the light emitted from the sample itself, this is more accurately described as luminescence, as the emitted light may be generated through fluorescence or phosphorescence, but the convention of the field will often use “Fluorescence Mode” to encompass both of these mechanistic possibilities.

The excitation wavelength was 390 nm, scanning emission data collected. The buffer employed was prepared using General Method A. The buffer composition was as follows: 250 mM 206, pH 6 (adjusted with sodium hydroxide), 50 mM 215, 50 mM 93, 50 mM 212 [avg. m.w.=6000]. The experimental protocol followed General Method C. The feed matrix employed was chicken scratch; detector compound was 50 at 10 μM, as specified. Each mycotoxin represented was tested as a separate sample, each at 10 μM; list of toxins includes: aflatoxin B1, citropten (a coumarin structurally-related to aflatoxins), zearalenone, β-zearalenol, «-zearalenol, ochratoxin A.

### Example 4

FIG. 3B is directly analogous to Example 3, showcasing toxin-selective signal production as controlled through detector compound and buffer formulation. This example is specific to ochratoxin A signal generation and quantification.

The data above is a graphical depiction of Fluorescence data with the toxin background subtracted. The excitation wavelength was 390 nm, scanning emission data collected. The buffer employed was prepared using General Method A. The buffer composition was as follows: 100 mM 206, pH 6 (adjusted with sodium hydroxide), 50 mM 213 [avg. m.w.=2000]. The experimental protocol followed General Method C. The feed matrix employed was chicken scratch; detector compound was 56 at 10 μM, as specified. Each mycotoxin represented was tested as a separate sample, each at 10 μM; list of toxins includes: aflatoxin B1, citropten (a coumarin struc-turally-related to aflatoxins), zearalenone, β-zearalenol, «-zearalenol, ochratoxin A.

### Example 5

FIGS. 4A and 4B provide evidence for the production of both absorbance and emission signals through the combination of a detector compound and a mycotoxin, namely deoxynivalenol, in the manner described in this invention. The increased absorbance in the presence of detector compound and the corresponding increased emission upon irradiation is representative of the ability of this invention to leverage ground-state manipulations for the generation of quantifiable emissive signals.

The data above are: Left) graphical depiction of Absorbance data with the detector compound background subtracted; Right) graphical depiction of Fluorescence data with the detector compound background subtracted; both data sets were measured from the same samples. For the Fluorescence mode data, the excitation wavelength was 450 nm, scanning emission data collected. The buffer employed was prepared using General Method A. The buffer composition was as follows: 125 mM 236, pH 8.5 (adjusted with hydrochloric acid), 125 mM 233, 16.66 mM 212 [avg. m.w.=6000]. The experimental protocol followed General Method C. The feed matrix employed was chicken scratch; detector compound was 92 at 10 μM; toxin was deoxynivalenol at 10 μM.

### Example 6

FIGS. 5A and 5B illustrate how Example 6 is directly analogous to Example 5, demon-strating the ability of this invention to create quantifiable emissive signal through selective manipulation of ground state absorbance profiles with the detector compounds and buffer formulations described herein. In this instance, zearalenone is the mycotoxin analyte of interest.

The data above are: Left) graphical depiction of Absorbance data with the detector compound background subtracted; Right) graphical depiction of Fluorescence data with the detector compound background subtracted; both data sets were measured from the same samples. For the Fluorescence mode data, the excitation wavelength was 425 nm, scanning emission data collected. The buffer employed was prepared using General Method A. The buffer composition was as follows: 250 mM 190, pH 10, (adjusted with sodium hydroxide), 25 mM 131, 50 mM 213 [avg. m.w.=2000]. The experimental protocol followed General Method C. The feed matrix employed was chicken scratch; detector compound was 115, 56, or 105, as specified, at 10 μM; toxin was zearalenone at 10 μM.

### Example 7

FIGS. 6A and 6B detail the emission data from two separate excitation modes as applied to samples containing either of two mycotoxins, namely aflatoxin and zearalenone. In this instance, the 390 nm excitation mode led to signal enhancement for aflatoxin without generating significant response from zearalenone, while the 450 nm excitation mode led to signal enhancement for zearalenone without generating significant response from aflatoxin. This data represents the potential for this invention to employ a single formulation of extraction buffer and detector compound to create quantifiable signal for multiple mycotoxins, with the signal specificity controlled through the method of irradiation.

The data above are: Left) graphical depiction of Fluorescence data with the detector compound background subtracted, excitation wavelength=390 nm; Right) graphical depiction of Fluorescence data with the detector compound background subtracted; excitation wavelength=450 nm. The buffer employed was prepared using General Method A. The buffer composition was as follows: 100 mM 206, pH 6 (adjusted with hydrochloric acid), 50 mM 213 [avg. m.w.=2000] with 20% DMSO. The experimental protocol followed General Method C. The feed matrix employed was chicken scratch; detector compound was 48 at 250 μM; mycotoxins employed were aflatoxin and zearalenone at 250 μM.

### Example 8

This example demonstrates the ability to calibrate and implement the detection technique described in this invention. Specifically, this example details the detection of ochratoxin A in corn. This example illustrates that this invention can provide quantitative detection of an agricul-turally-relevant mycotoxin in a major feed/food ingredient at commercially-relevant levels.

| | |
|---|---|
| Toxin Conc | Signal Data |

| nM | ppb | ΔEm | SD | S/N |
|---|---|---|---|---|
| 0.0 | 0.0 | 13 | 0.8% | . |
| 3.7 | 6.0 | 138 | 0.6% | 1.3% |
| 11 | 18 | 110 | 0.8% | 1.0% |
| 33 | 54 | 409 | 0.8% | 3.8% |
| 100 | 162 | 1491 | 1.0% | 13.9% |
| 300 | 485 | 3880 | 0.5% | 36.3% |

The data above is presented in two formats: Left) graphical depiction of Fluorescence mode data with detector compound background subtracted for the calibration curve of interest; Right) table of calibration curve measurements and quantitative performance metrics. Specific performance metrics are: ΔEm (measured metric that is directly correlated with toxin concentration), standard deviation of measurement, S/N. The Fluorescence mode data was collected with an excitation wavelength of 385 nm; the quantified emission data was collected at 450 nm, using data points at 510 nm, 550 nm, and 590 nm to aid the background subtraction efforts. The buffer employed was prepared using General Method A. The buffer composition was as follows: 500 mM 199, pH 8 (adjusted with hydrochloric acid), 100 mM 189, 35 mM 12, 15 mM 212 [avg. m.w.=6000]. The experimental protocol followed General Method E. The feed matrix employed was corn; detector compound was 48 at 10 μM; mycotoxin employed was ochratoxin at variable concentrations, as specified in the data table.

### Example 9

This example demonstrates the ability to calibrate and implement lateral flow device immunoassay technologies in conjunction with the extraction buffer technologies detailed within this invention. Specifically, this example details the detection of aflatoxin in corn as well as other feed/food matrices. This example illustrates the functional performance achievable when employing the extraction buffers described in this invention in conjunction with immunoassay technologies. Further, this example demonstrates the ability to provide quantitative detection of an agriculturally-relevant mycotoxin at commercially-relevant concentrations in several substrates of interest at a level that matches or exceeds that of the current state-of-the-art.

The data above is presented in two formats: Left) graphical depiction of measured concentration readout from Raptor reader instrument for multiple buffer formulations; Right) variance graph of curve-adjusted concentration measurements for buffer formulations employed in multiple feed matrices. The variance plot is intended to highlight the consistency of readout across feed types (ideally centering at the prescribed 10 ppb level), thus Buffer A is nearly equivalent to the state-of-the-art (Reveal Q+ Max) and Buffer C is arguably better. The buffers employed were prepared using General Method A, with Neogen's Reveal Q+ Max buffer serving as a reference point. The novel buffer compositions were as follows: A) 500 mM 195, 100 mM 210, pH 8.0 (adjusted with potassium hydroxide), 50 mM 93, 12.5 mM 213 [avg. m.w.=2000]; B) 450 mM 201, 100 mM 198, pH 7.5 (adjusted with potassium hydroxide), 50 mM 93, 12.5 mM 213 [avg. m.w.=2000]; C) 400 mM 202, 100 mM 198, pH 7.5 (adjusted with potassium hydroxide), 12.5 mM 213 [avg. m.w.=2000]. The experimental protocol followed General Method F. Calibration curve data was collected in corn; the toxin employed was aflatoxin B1; the curve, as displayed above, is the data provided from the Neogen Raptor reader device, which is pre-cali-brated to the analyte responsiveness of the assay in the commercial buffer formulation. The variance data was collected in the following matrices: corn, corn gluten meal, corn gluten feed, wheat, corn silage, and a trout feed [Rangen; “aqua feed”]; the mycotoxin employed was aflatoxin B1 at 10 ppb; the concentration data was calculated from the measured ratio of control line intensity to test line intensity (“Ratio”), using the logistic function generated for each buffer via non-linear regression of calibration curve data to convert measured Ratios to Concentration on a buffer-by-buffer basis.

### Example 10

This example demonstrates the ability to implement the extraction buffers described in this invention in conjunction with lateral flow device technologies to detect naturally-occurring mycotoxins, specifically aflatoxins in corn samples. This example illustrates the concepts com-municated in this invention regarding the use of extraction buffers to solubilize naturally-occurring mycotoxins while facilitating quantitative detection with immunoassay techniques.

| | No Tox | | | +Tox [Natural] (9.3 ppb AF) | | |
|---|---|---|---|---|---|---|
| Extraction Buffer | Control | Test | Ratio | Control | Test | Ratio |
| Reveal Q+ Max | 266918 | 599237 | 2.26 | 518518 | 465672 | 0.90 |
| A | 205318 | 605222 | 2.96 | 375819 | 439067 | 1.17 |
| B | 193684 | 628709 | 3.25 | 396628 | 487671 | 1.23 |
| C | 171053 | 673199 | 3.94 | 350071 | 604775 | 1.72 |
| D | 201976 | 617895 | 3.06 | 251414 | 506313 | 2.03 |
| E | 217824 | 633342 | 2.92 | 322864 | 588362 | 1.84 |
| F | 203409 | 647457 | 3.19 | 325108 | 550592 | 1.70 |
| G | 212316 | 679731 | 3.22 | 383535 | 589728 | 1.58 |
| H | 220180 | 651441 | 2.97 | 328529 | 571089 | 1.74 |
| I | 214989 | 634939 | 2.96 | 328791 | 568922 | 1.73 |
| J | 178651 | 569950 | 3.25 | 288586 | 513430 | 1.78 |
| K | 206944 | 615536 | 2.98 | 309564 | 567983 | 1.84 |
| L | 202140 | 645305 | 3.20 | 390052 | 539197 | 1.38 |

The tabular data above represents the measured line intensities (control line and test line) for lateral flow devices (Neogen's Reveal Q+ Max test strips) as measured with Neogen's Raptor reader instrument. The Ratio data is the quotient of test line and control line data; as described above, Ratio data may be employed to calculate analyte concentration when the corresponding logistic function calibration curve is available; any reduction of Ratio in response to the presence of analyte is indicative of functional performance. The buffers employed were prepared using General Method A, with Neogen's Reveal Q+ Max buffer serving as a reference point. The novel buffer compositions were as follows: A-C) same formulations as Buffers A-C in Example 11; D) 500 mM 195, 100 mM 210, pH 8.0 (adjusted with potassium hydroxide), 35 mM 233, 12.5 mM 213 [avg. m.w.=2000]; E) 450 mM 201, 100 mM 198, pH 7.5 (adjusted with potassium hydroxide), 35 mM 242, 12.5 mM 213 [avg. m.w.=2000]; F) 450 mM 200, 100 mM 198, pH 7.5 (adjusted with potassium hydroxide), 35 mM 242, 12.5 mM 213 [avg. m.w.=2000]; G) 400 mM 202, 100 mM 198, pH 7.5 (adjusted with potassium hydroxide), 35 mM 242, 12.5 mM 213 [avg. m.w.=2000]; H) 450 mM 201, 100 mM 198, pH 7.5 (adjusted with potassium hydroxide), 12.5 mM 213 [avg. m.w.=2000]; I) 450 mM 201, 100 mM 198, pH 7.5 (adjusted with potassium hydroxide), 35 mM 242, 35 mM 233, 12.5 mM 213 [avg. m.w.=2000]; J) 500 mM 195, 100 mM 210, pH 8.0 (adjusted with potassium hydroxide), 50 mM 215, 12.5 mM 213 [avg. m.w.=2000]; K) 450 mM 201, 100 mM 198, pH 7.5 (adjusted with potassium hydroxide), 50 mM 215, 12.5 mM 213 [avg. m.w.=2000]; L) 450 mM 200, 100 mM 198, pH 7.5 (adjusted with potassium hydroxide), 50 mM 93, 12.5 mM 213 [avg. m.w.=2000]. The experimental protocol followed General Method F. The feed matrix employed was corn; mycotoxin analyte was aflatoxin; corn samples were acquired from Trilogy Labs with experimentally-validated aflatoxin level (No Tox=no detectable amount [i.e. ≤1.0 ppb], +Tox=9.3 ppb).

### Example 11

FIG. 8A demonstrates the increased magnitude of absorbance and shifted Abs maxima upon introduction of detector compounds in the presence and absence of various mycotoxins. This data is representative of the alteration to ground-state spectroscopic behavior achievable with this invention.

The data above is a graphical depiction of Absorbance data with detector compound subtracted and toxin-specific contributions to ΔEm also subtracted. The buffer employed was prepared using General Method A. The buffer composition was as follows: 100 mM 215, pH 6 (adjusted with potassium hydroxide), 15 mM 213 [avg. m.w.=2000]. The experimental protocol followed General Method C. The feed matrix employed was chicken scratch [5-grain blend comprised of corn, millet, barley, wheat, and sunflower seed; Dumor]; detector compounds were 166, 98, or 47 at 250 μM, as specified; toxins were aflatoxin or zearalenone at 250 μM.

### Example 12

FIG. 8B is analogous to the experiment detailed in Example 3, but it was performed in corn silage, serving as evidence that the invention detailed within this report is operative in complex, farm-made feeds that are relevant to livestock production.

The data above is a graphical depiction of Absorbance data with detector compound subtracted and toxin-specific contributions to ΔEm also subtracted. The buffer employed was prepared using General Method A. The buffer composition was as follows: 250 mM 190, pH 8.5 (adjusted with hydrochloric acid), 1.25 mg/mL pectinase, 100 mM 137, 15 mM 213 [avg. m.w.=2000]. The experimental protocol followed General Method C. The feed matrix employed was corn silage; detector compounds were 125 or 168 at 10 μM, as specified; toxin was ochratoxin A at 1.0 M.

Analysis and quantification of the extracted analytes can be performed by any number of methods, with or without any pre- or post-extraction manipulations. Analyses may include, but are not limited to UV/V is spectroscopy, IR-based spectroscopy, X-ray spectroscopy, nuclear magnetic resonance spectroscopy, fluorimetry (aka. fluorometry) or any other suitable luminescence-based method, mass spectrometry of all forms, elemental analysis, atomic absorption or atomic emission spectroscopy, electrochemical analyses or electrophoresis, optical detection methods, magnetism-based methods, titration, gravimetric analyses, nephelometry, or any form of qualitative assessment or analysis.

In addition to methods in which analytes are extracted, these buffer compositions are also viable options for alternative uses of buffer solutions. This may include, but is not limited to, biological or chemical assays, cell or tissue cultures, storage of small molecules or macromolecules or cells or tissues, biological or chemical production or other purification processes.

A brief description detailing the structural and functional features used to design each of the buffer component classes is provided below. These definitions are preferred for this research, and therefore may also match broader scientific definitions. The following descriptions assume that each component would be in an aqueous solution, but analogous function is also possible in non-aqueous environments.

First, a buffer utilized in the present invention is defined as a species that stabilizes the pH in the medium. Such a buffer is represented by small molecules comprised of an acidic component, for example carboxylic acid, and/or a basic functionality such as an amine with a pK_{A} of the acid or the conjugate acid within the range of 3-12, preferably having a range between 5-10 with a concentration of buffering components generally between 100-500 mM.

Second, a co-solvent is defined as a species that facilitates the solubilization of a target mycotoxin during extraction and improves flow rate during filtration. Such a co-solvent is represented by small molecules or polymers that are water-soluble but are to some degree lipophilic, thus enabling extraction of ambiphilic mycotoxins. Ethylene glycol derivatives have proven useful for this purpose; operating concentrations generally between 10-50 mM.

Third, a useful component is a digestion enzyme defined as an enzyme that digests certain plant-derived materials. Such a digestion enzyme is represented by isolated enzymes, typically those that decompose carbohydrates including functional products such as pectinase and cellulase with operating concentrations generally between 0.1-2.5 mg/mL.

Next, another useful component is a surfactant defined as an ambiphilic species capable of solubilizing lipophilic species in an aqueous environment; represented by small molecules comprised of a polar functionality where carboxylic acid, or sulfonic acid are introduced as an acid or a salt, along with a lipophilic functionality such as linear, branched, or (poly) cyclic aliphatic groups; hydrocarbon functional groups lacking highly polar functional groups including camphorsulfonic acid are promising examples operating as concentrations generally between 10-150 mM.

Further, another useful component includes viscosity agents, which are defined as a species that alters the viscosity of the extraction medium to improve filtration flow rate and/or signal production. Examples of useful viscosity agents include small molecules or polymers that are water-soluble, typically containing many hydroxyl groups and other polar functionality on a carbon backbone. Non-reducing sugars such as trehalose have proven useful thus far in operating concentrations generally between 10-150 mM.

Chelators are sometimes useful, being defined as a species that sequesters materials within the extraction medium to reduce background noise, where such chelators are represented by small molecules or polymers comprised of polydentate polar functionality (e.g. polyols, polyamines, aminoalcohols, guanidines, bypyridines) that can form multiple bonds to a given background contaminant, be it metal ions or small molecule chromophores. Chitosan is a polymeric material that has proven useful thus far at operating concentrations generally between 0.25-500 mM.

Redox modulators have proven to be useful. They are defined as a species that manipulates the oxidizing and reducing capacity of the extraction medium, such as those represented by small molecules comprised of redox active functionality, such as H-atom donors or sulfur-based functional groups (e.g. thiols, disulfides, thioesters, dithioesters, thioamides). In particular, rhodanine derivatives, aldrithiol, penicillamine, and maltol derivatives have proven promising thus far at operating concentrations generally between 10-150 mM.

Yet another useful component is a photochemistry promoter defined as a species that manipulates the extraction medium to promote signal production through an undefined mechanism; represented by small molecules, typically heteroaromatic systems, that do not match the requirements to be a detector compound but are suspected to stabilize the ground state and/or excited state complexes in order to improve quantum yield. Particularly useful components include saccharin and purine-like bases in operating concentrations generally between 10-150 mM.

Finally, a salt may be incorporated that is defined as a species comprised of simple ionic components meant to provide the appropriate osmotic pressure for the other components to best perform their function and/or to facilitate buffer preparation (i.e. dictate pH). Useful salts are represented by alkali and alkaline earth salts with simple counterions, especially such as sodium borate in operating concentrations generally between 10-150 mM.

As there are structural analogies between certain candidates in the buffer, co-solvent, surfactant, viscosity agent, and chelator categories, certain examples may thereafter become re-categorized. The redox modulators, photochemistry promoters, and salts may also perform additional functions, such as chelation, or viscosity manipulation. These category labels are used for internal design and optimization platforms, thus a given component in a final commercial buffer composition may perform multiple roles and multiple functions. Ultimately, the structural identity of each component and the full composition of the buffer will dictate the overall function of the extraction buffer, rather than by simple categorical labels.

### Minimum Functional Need:

Only a few core components from the above list are required to enter the desired signal production modes (see Examples section above). The additional reagents are meant to refine and improve the signal, and the final composition could require any number of potential additives and additive classes. Preferred features include a buffer defined as a species that stabilized the pH in the medium with a co-solvent defined as a species that facilitates the solubilization of the mycotoxin during extraction and improves flow rate during filtration.

However, for minimum functional need, only a single component is necessary to achieve the analytical functions detailed, namely the buffer, defined as a species that stabilizes the pH in the medium. The additional reagents are meant to refine and improve the efficacy, while the extraction efficiency, removal of unwanted background components signal, the ability to integrate with existing protocols for LFDs or other technologies, and any other function that is associated with performance in the detection methodology is intended as a function downstream of buffer usage.

### Generic Depictions of Buffer Components:

A generic depiction of each envisioned buffer component class is shown below.

### Specific Buffer Components

Buffer Components
Basic and Zwitterionic Functionality (e.g. amino acid derivatives)

| | |
|---|---|
| 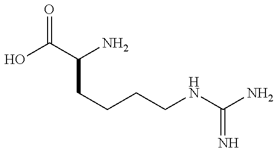 [SMILES: N=C(N)NCCCC[C@H](N)C(=O)O] | S30 |
| [D, L, or DL] | |
| 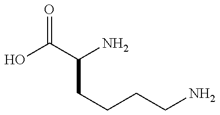 | S31 |
| [D, L, or DL] | |
| 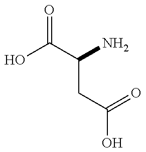 | S40 |
| [D, L, or DL] | |
| 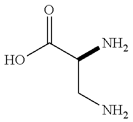 | S41 |
| [D, L, or DL] | |
| 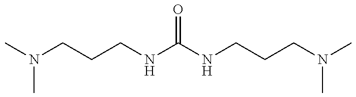 | |
| bis-dimethylaminopropyl urea | |
| [and salts] | |
| 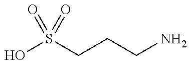 | P73 |
| [and salts] | |
| 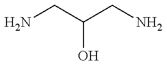 | R02 |
| [and salts] | |
| 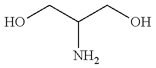 | R03 |
| [and salts] | |
| 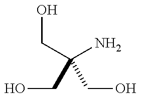 | R01 |
| [and salts] | |
| 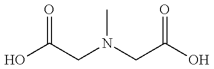 | Q04 |
| [and salts] | |
| 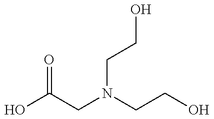 [SMILES: O=C(O)CN(CCO)CCO] | Q02 |
| [and salts] | |
| 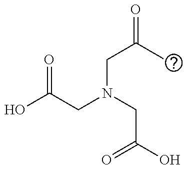 | Q03 |
| [and salts] | |
| 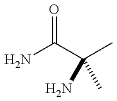 | R20 |
| [D, L, or DL; and salts] | |
| 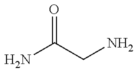 | R25 |
| [D, L, or DL; and salts)] | |
| 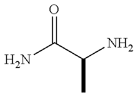 | R21 |
| [D, L, or DL; and salts)] | |
| 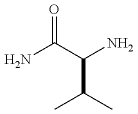 | R26 |
| [D, L, or DL; and salts)] | |
| 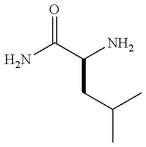 | R22 |
| [D, L, or DL; and salts)] | |
| 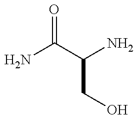 | R24 |
| [D, L, or DL; and salts)] | |
| 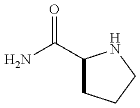 | R23 |
| [D, L, or DL; and salts)] | |

Acidic Functionality [including salts]

| | |
|---|---|
| 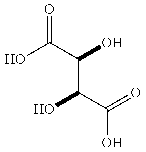 | S01 |
| [D, L, or DL] | |
| 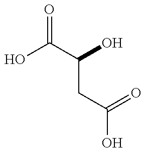 | S04 |
| [R, S, or RS] | |
| 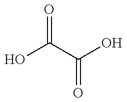 | S06 |
| 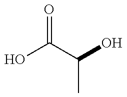 | S07 |
| [R, S, or RS] | |
| 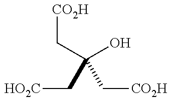 | S03 |

[symbol]

pectinase [aka pectic enzyme] brand: LD Carlson
cellulase brand: MarkNature

[symbol]

| | |
|---|---|
| 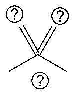 | Z01 |
| 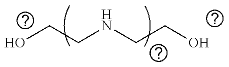 | [symbol] |
| [symbol] | |
| 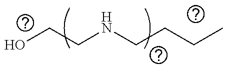 | P02 |
| [symbol] | |

[symbol]

| | |
|---|---|
| 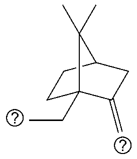 | B71 |
| 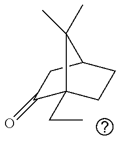 | S70 |
| 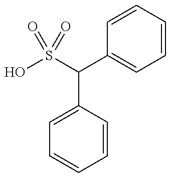 [SMILES: O=S(=O)(O)C(c1ccccc1)c1ccccc1] | S80 |
| 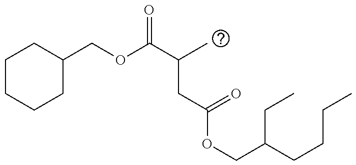 [SMILES: CCCCC(CC)COC(=O)CC(C)C(=O)OCC1CCCCC1] | P70 |
| 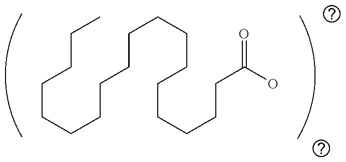 | P71 |
| 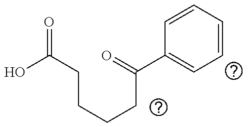 [SMILES: O=C(O)CCCCC(=O)c1ccccc1] | [symbol] |
| [and salts] | |

[symbol]

Agents

| | |
|---|---|
| 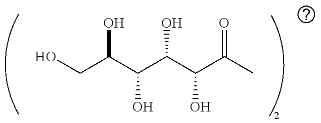 | P42 |
| [and other salts] | |
| 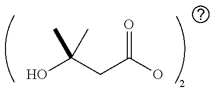 | Q41 |
| [and other salts] | |
| 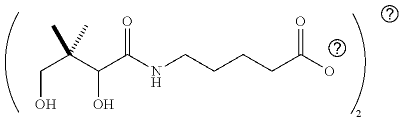 | Q40 |
| [and other salts][D, L, or DL] | |
| 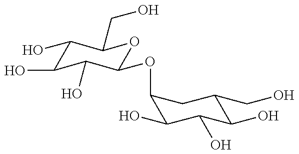 [SMILES: OCC1CC(OC2OC(CO)C(O)C(O)C2O)C(O)C(O)C1O] | P20 |
| 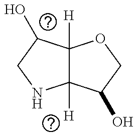 | P41 |
| [D, L, or DL; and related derivatives] | |
| 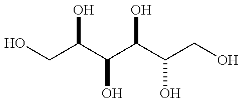 [SMILES: OC[C@@H](O)[C@H](O)[C@@H](O)[C@@H](O)CO] | P40 |
| [D, L, or DL] | |
| 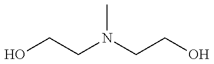 | R50 |
| 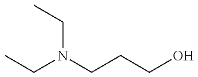 | R51 |
| 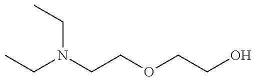 | R62 |
| 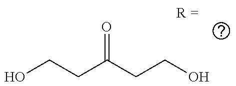 | |
| [symbol] | |
| 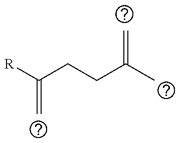 | |
| [symbol] | |
| 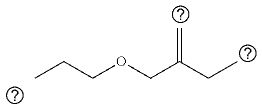 | |

[symbol]

| | |
|---|---|
| 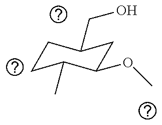 | |
| Q80 and Q81 | |
| 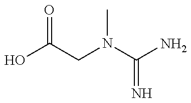 | |
| creatine | |
| 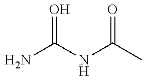 | |
| N-acetyl guanidine | |
| 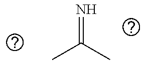 | |
| guanidine carbonate | |
| 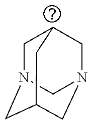 | R50 |
| 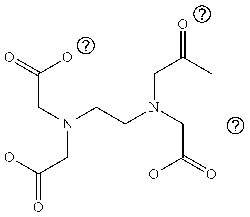 [SMILES: CC(=O)CN(CCN(CC(=O)O)CC(=O)O)CC(=O)O] | Q01 |
| [and other salts] | |

[symbol]

| | |
|---|---|
| 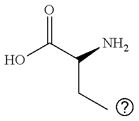 | S60 |
| [D, L, or DL] | |
| 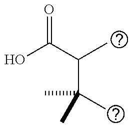 | S51 |
| [D, L, or DL] | |
| 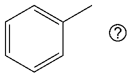 | F06 |
| [and other salts] | |
| 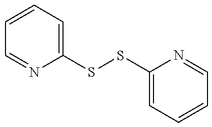 [SMILES: c1ccc(SSc2ccccn2)nc1] | F05 |
| 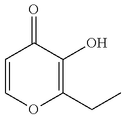 | |
| [symbol] | |
| 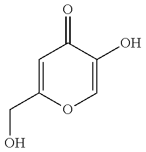 | K06 |
| 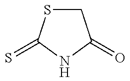 | H19 |
| 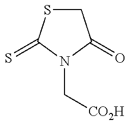 [SMILES: O=C(O)CN1C(=O)CSC1=S] | H32 |
| [and salts] | |
| 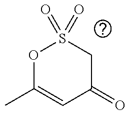 | S52 |
| [and other salts] | |
| 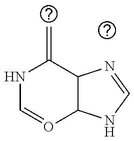 | C04 |

[symbol]

| | |
|---|---|
| 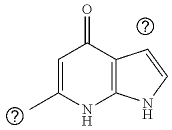 | B01 |
| 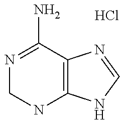 | B02 |
| 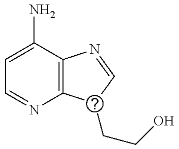 [SMILES: Nc1ccnc2c1N=CC2CCO] | B10 |
| 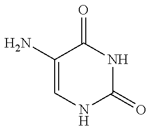 | C08 |
| 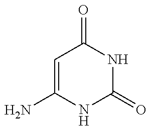 | C10 |
| 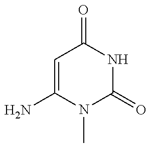 | C02 |
| 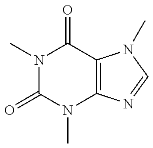 | C04 |
| 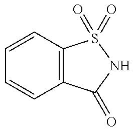 [SMILES: O=C1NS(=O)(=O)c2ccccc21] | H20 |
| [and salts] | |
| 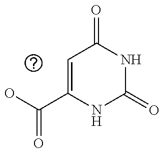 [SMILES: O=C(O)c1cc(=O)[nH]c(=O)[nH]1] | C20 |
| [and other salts] | |
| 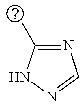 | H03 |
| 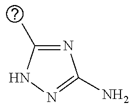 | H04 |
| 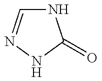 | H05 |
| 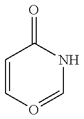 | C07 |
| 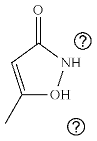 | C03 |
| 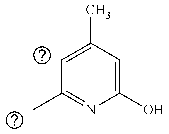 | C17 |
| 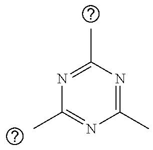 | C15 |
| 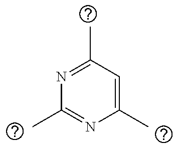 | |
| melamine | |

[symbol]

MgO
magnesium oxide
CaO
calcium oxide
ZnO
zinc oxide
MgSO₄
magnesium sulfate
Na [symbol]
sodium borate
Na₂B₄O₇
sodium tetraborate [“Borax”]

[symbol]

indicates data missing or illegible when filed

### Generic Depictions of Detector Compounds:

A generic depiction of each subclass of detector compound is shown in the molecular models below. The models provide a range of complexity levels i.e. highly generic at the top and intermediate complexity at the bottom for certain subclasses of interest.

Generic Detector Compounds

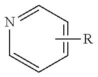

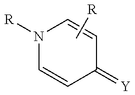

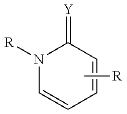

pyridines and pyridinones

[symbol]

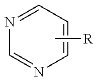

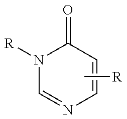

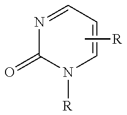

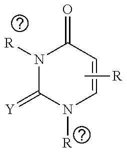

pyrimidines and pyrimidinones

[symbol]

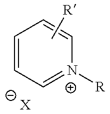

pyridinium salts
R,R = any;
X = OR

[symbol]

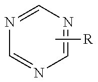

triazine
R = any

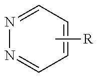

triazine
R = any

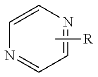

pyridazine
R = any

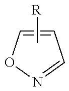

isoxazoles
R,R = any

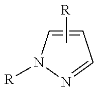

pyrazoles
R,R = any

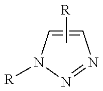

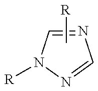

triazoles
R,R = any

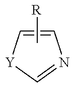

oxazole, thiazole, imidazole, etc.
R = any; Y = O, S, SO₂NR

[symbol]

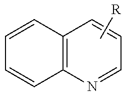

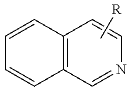

qionolines and isoquinolines
R,R = any
Y = O, S, SO₂NR, CH, CHR, CR [symbol] ;
Z = N, CR

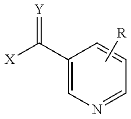

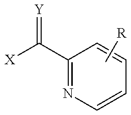

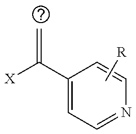

nicotinic, picolinic, and isopicolic acid derivative [symbol]
R,R = any;
X = OR, SR, NHR, NR halide, CR₃
Y = O, S, NR, CH₃, CHR, CR₃

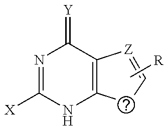

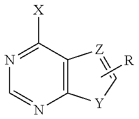

purine bases and mimics
R,R = any;
X = OR, SR, NHR, NR halide
Y = O, S, SO₂, NR; Z = N, CR

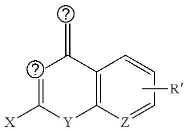

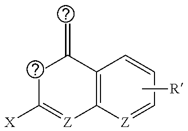

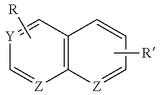

fused pyrimidines and related structures
R,R = any;
X = OR, SR, NHR, NR, halide [symbol]
Y = O, S, SO₂, NR; Z = N, CR

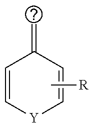

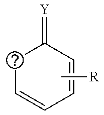

pyrones and related compound
R = any;
Y = O, S, NR, CH₃, CHR, CR₃

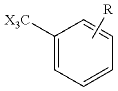

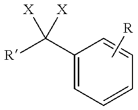

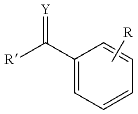

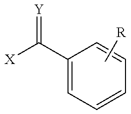

benzene derivatives
R,R = any;
X = OR, SR, NHR, NR halide, CR₃
Y = O,S, NRCH₃, CHR, CR₃

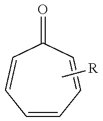

tropoiones
R = any

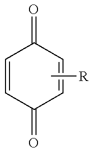

quinones
R = any

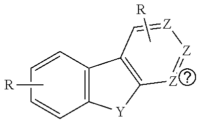

carbazoles and derivatives
R,R = any;
Y = O, S, SO2, NR, CH, CHR, CR₃;
Z = N,CR

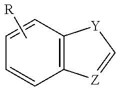

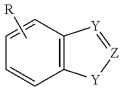

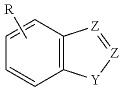

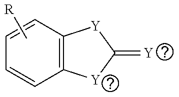

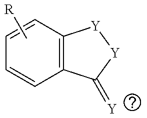

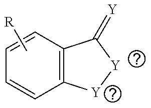

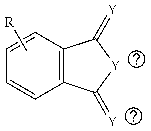

benzo-fused azoles, azolinones, and azolidinones
R,R = any; Y = O, S, SO₂, NR, CH₃, CHR, CR₃; Z = N,CR

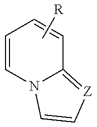

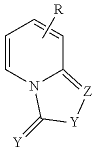

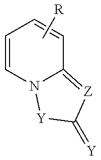

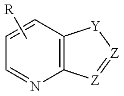

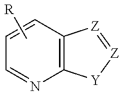

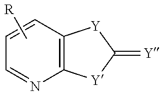

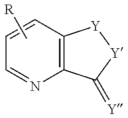

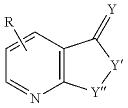

fused pyridines
R,R = any; Y = O, S, SO₂, NR, CH₃, CHR, CR₃; Z = N,CR [symbol]
indicates data missing or illegible when filed

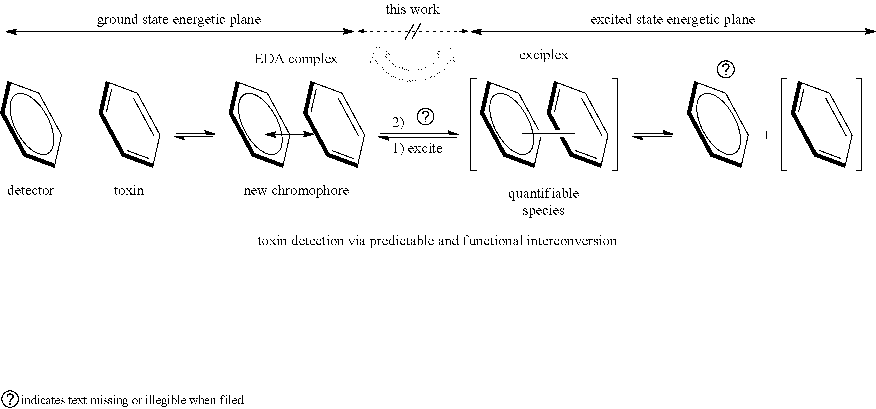

Of note, any given detector compound and/or buffer conditions can be used for more than one analyte. The photochemical behavior is (in its ideal form) independent of other photochemical mechanisms operating in the same mixture, thus quantification of a given analyte can be done contemporaneously with the quantification of multiple other toxins in the same conditions.

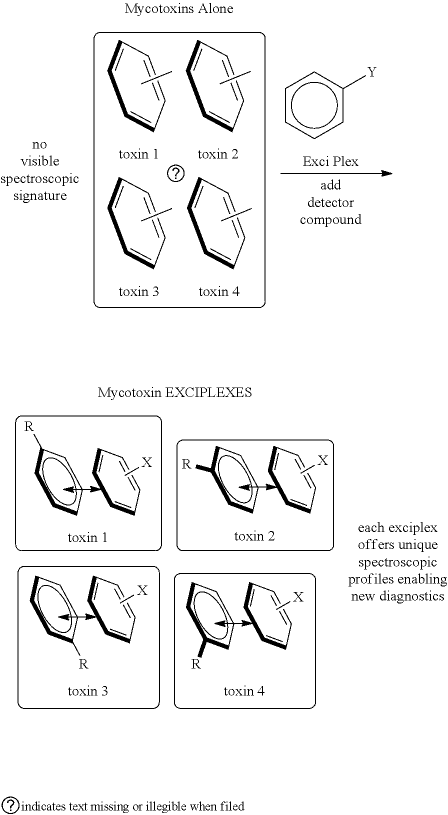

### Specific Detector Compounds:

Canonical Acceptors

| | |
|---|---|
| 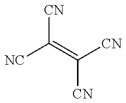 | A01 |
| 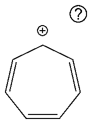 | A02 |
| 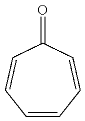 | A03 |
| 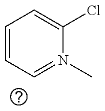 | A04 |
| 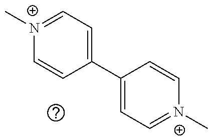 | A05 |
| 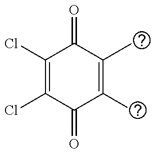 | A06 |
| 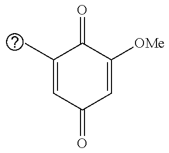 | A07 |
| 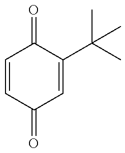 | A08 |

DNA Bases and Base Derivates Mimics

| | |
|---|---|
| 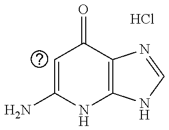 [SMILES: Nc1cc(=O)c2nc[nH]c2[nH]1 ; Cl] | B01 |
| 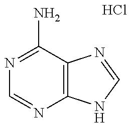 | B02 |
| 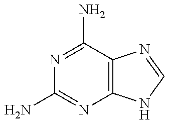 [SMILES: Nc1nc(N)c2nc[nH]c2n1] | B03 |
| 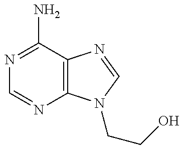 [SMILES: Nc1ncnc2c1ncn2CCO] | B10 |
| 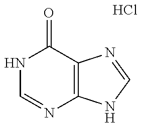 | C14 |
| 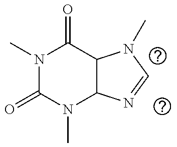 | C01 |
| 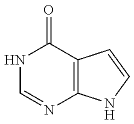 | C12 |
| 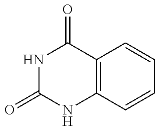 [SMILES: O=c1[nH]c(=O)c2ccccc2[nH]1] | C11 |
| 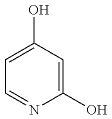 | [symbol] |
| 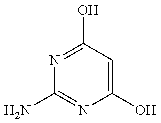 | C12 |
| 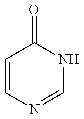 | C07 |
| 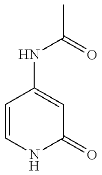 | B08 |
| 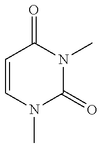 | B06 |
| 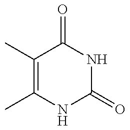 | B07 |
| 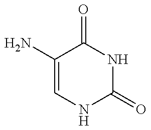 | C08 |
| 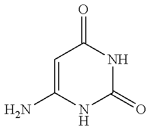 | C10 |
| 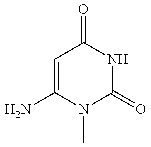 | C02 |
| 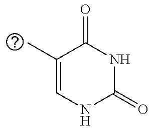 | B03 |
| 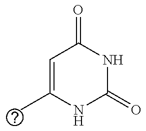 | B04 |
| 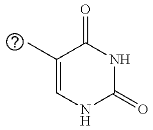 | B05 |
| 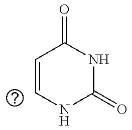 | H07 |
| 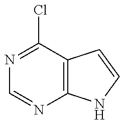 | C14 |
| 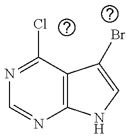 [SMILES: Clc1ncnc2[nH]cc(Br)c12] | C16 |
| 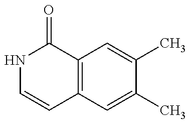 | C13 |
| 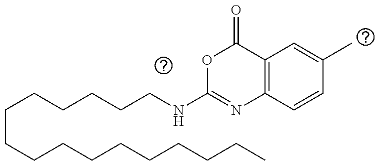 [SMILES: CCCCCCCCCCCCCCCCNc1nc2ccc(C)cc2c(=O)o1] | D24 |
| 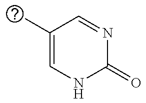 | C06 |
| 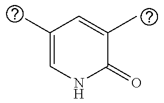 | H26 |
| 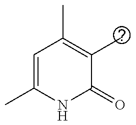 | B16 |
| 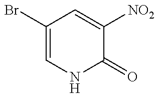 [SMILES: O=c1[nH]cc(Br)cc1[N+](=O)[O-]] | G06 |
| 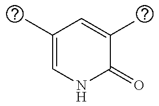 | G04 |
| 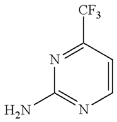 [SMILES: Nc1nccc(C(F)(F)F)n1] | C46 |
| 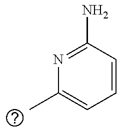 | C15 |
| 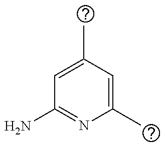 | C16 |
| 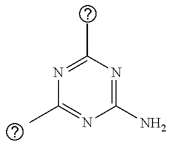 | C19 |
| 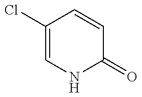 | H11 |
| 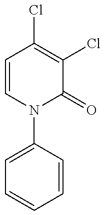 [SMILES: O=c1c(Cl)c(Cl)ccn1-c1ccccc1] | H12 |
| 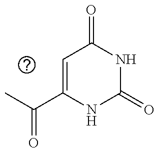 | C20 |

[symbol]

| | |
|---|---|
| 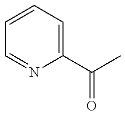 | E01 |
| 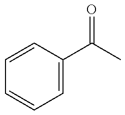 | E02 |
| 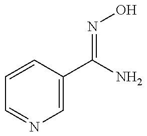 | E03 |
| 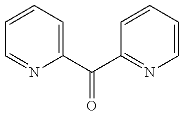 [SMILES: O=C(c1ccccn1)c1ccccn1] | E06 |
| 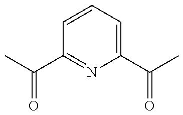 | E04 |
| 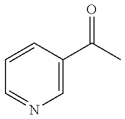 | H43 |
| 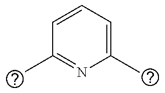 | [symbol] |
| 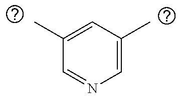 | E08 |
| 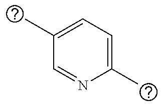 | E09 |
| 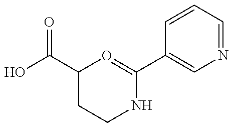 | E14 |
| 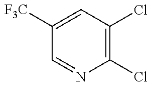 [SMILES: FC(F)(F)c1cnc(Cl)c(Cl)c1] | H08 |
| 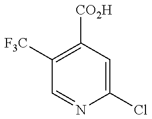 [SMILES: O=C(O)c1cc(Cl)ncc1C(F)(F)F] | [symbol] |
| 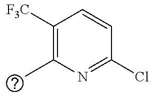 | H27 |
| 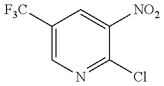 [SMILES: O=[N+]([O-])c1cc(C(F)(F)F)cnc1Cl] | H28 |
| 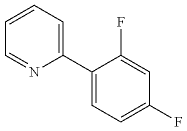 [SMILES: Fc1ccc(-c2ccccn2)c(F)c1] | H18 |
| 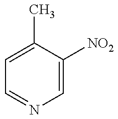 | G03 |
| 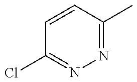 | H10 |
| 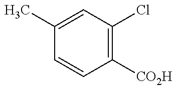 | H34 |
| 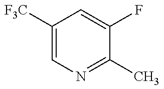 | H29 |
| 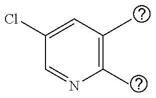 | G04 |
| 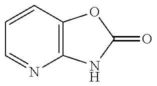 | D22 |
| 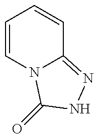 | [symbol] |
| 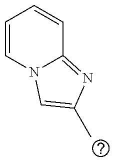 | G08 |
| 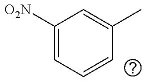 | G12 |
| 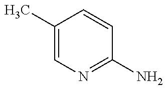 | H40 |

[symbol]

| | |
|---|---|
| 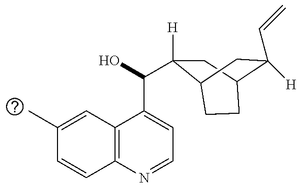 [SMILES: C=CC1CC2CCC1CC2[C@H](O)c1ccnc2ccc(C)cc12] | D16 |
| 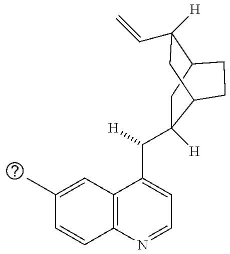 | G28 |
| 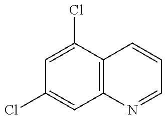 [SMILES: Clc1cc(Cl)c2cccnc2c1] | D16 |
| 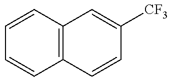 [SMILES: FC(F)(F)c1ccc2ccccc2c1] | D18 |
| 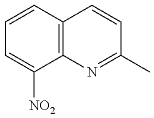 | D07 |
| 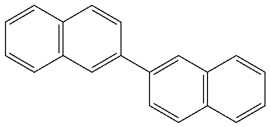 [SMILES: c1ccc2cc(-c3ccc4ccccc4c3)ccc2c1] | D25 |
| 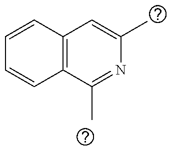 | D26 |
| 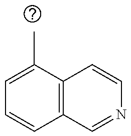 | D06 |
| 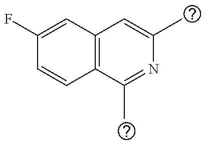 | G29 |
| 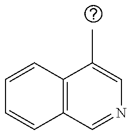 | D36 |
| 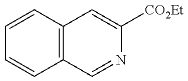 [SMILES: CCOC(=O)c1cc2ccccc2cn1] | G01 |
| 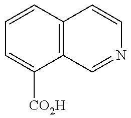 [SMILES: O=C(O)c1cccc2ccncc12] | G02 |
| 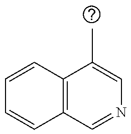 | D30 |

Additional Benzo-fused

[symbol]

| | |
|---|---|
| 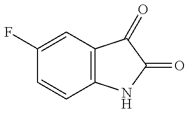 [SMILES: O=C1Nc2ccc(F)cc2C1=O] | D01 |
| 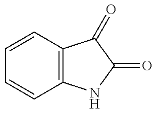 [SMILES: O=C1Nc2ccccc2C1=O] | D02 |
| 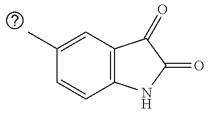 | D03 |
| 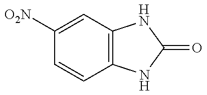 [SMILES: O=c1[nH]c2ccc([N+](=O)[O-])cc2[nH]1] | D04 |
| 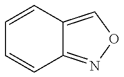 | D18 |
| 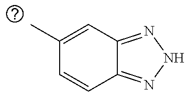 | D17 |
| 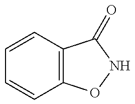 | D14 |
| 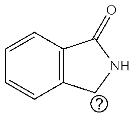 | D16 |
| 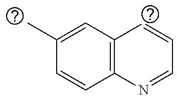 | D06 |
| 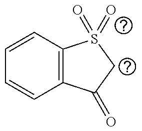 [SMILES: O=C1CS(=O)(=O)c2ccccc21] | H20 |
| 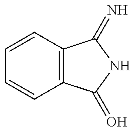 [SMILES: N=C1NC(=O)c2ccccc21] | H30 |
| 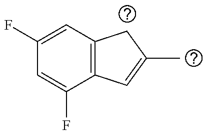 | H02 |
| 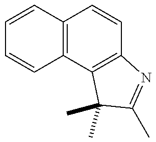 | D25 |
| 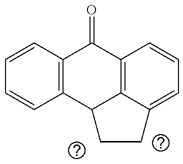 [SMILES: O=C1c2ccccc2C2CCc3cccc1c32] | A09 |
| 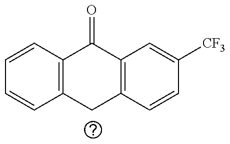 [SMILES: O=C1c2ccccc2Cc2ccc(C(F)(F)F)cc21] | G13 |
| 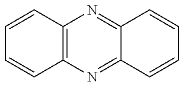 [SMILES: c1ccc2nc3ccccc3nc2c1] | D27 |
| 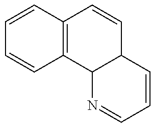 [SMILES: C1=CC2C=Cc3ccccc3C2N=C1] | G25 |
| 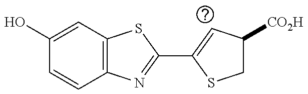 [SMILES: O=C(O)[C@@H]1C=C(c2nc3ccc(O)cc3s2)SC1] | K01 |
| 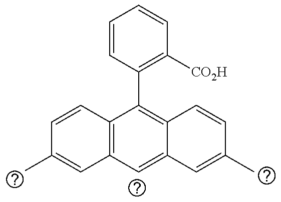 | K02 |
| [various salts] | |
| 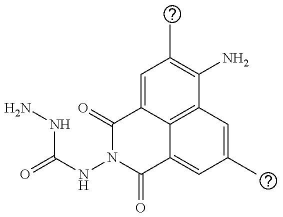 [SMILES: Cc1cc2c3c(cc(C)c(N)c3c1)C(=O)N(NC(=O)NN)C2=O] | K03 |
| 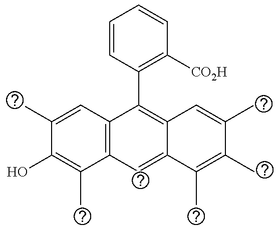 | K04 |
| [various salts] | |

[symbol]

| | |
|---|---|
| 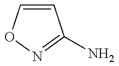 | H32 |
| 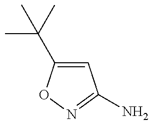 | H01 |
| 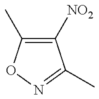 | G16 |
| 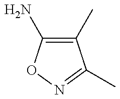 | H23 |
| 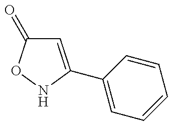 [SMILES: O=c1cc(-c2ccccc2)[nH]o1] | H24 |
| 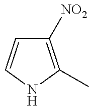 | G13 |
| 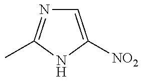 | G05 |
| 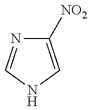 | G09 |
| 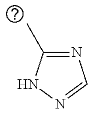 | H03 |
| 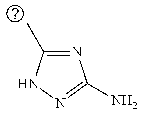 | H04 |
| 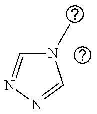 | H05 |
| 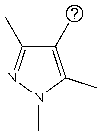 | S07 |
| 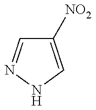 | G07 |
| 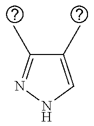 | H07 |
| 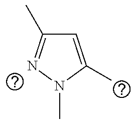 | H13 |
| 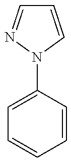 [SMILES: c1ccc(-n2cccn2)cc1] | H14 |
| 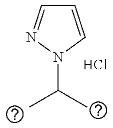 | H15 |
| 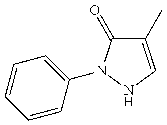 | H25 |
| 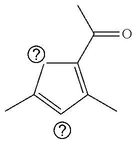 | S06 |
| 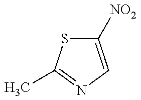 | G10 |
| 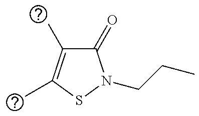 | H21 |
| 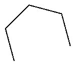 | |
| 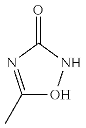 | C03 |
| 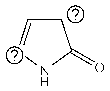 | B46 |
| 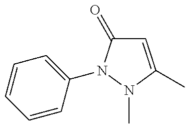 | B37 |
| 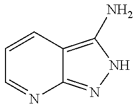 | D23 |
| 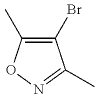 | H28 |

[symbol]

| | |
|---|---|
| 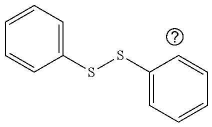 [SMILES: c1ccc(SSc2ccccc2)cc1] | F05 |
| 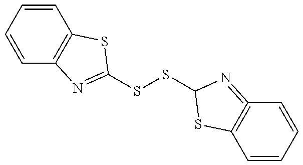 | F08 |
| 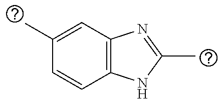 | G11 |
| 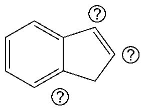 | E12 |
| 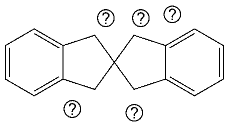 [SMILES: c1ccc2c(c1)CC1(C2)Cc2ccccc2C1] | F08 |
| 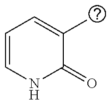 | X09 |
| 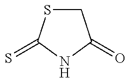 | H17 |
| 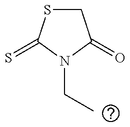 | H32 |
| 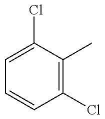 | F01 |
| 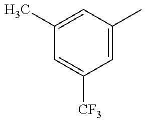 | F02 |
| 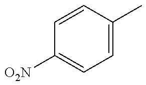 | F03 |
| 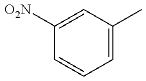 | F04 |
| 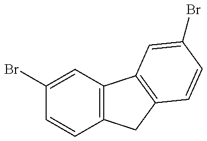 [SMILES: Brc1ccc2c(c1)-c1cc(Br)ccc1C2] | D11 |

[symbol]

| | |
|---|---|
| 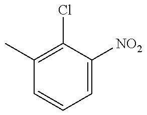 | G01 |
| 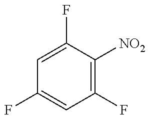 [SMILES: O=[N+]([O-])c1c(F)cc(F)cc1F] | G02 |
| 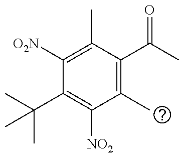 [SMILES: CC(=O)c1c(C)c([N+](=O)[O-])c(C(C)(C)C)c([N+](=O)[O-])c1C] | G14 |
| 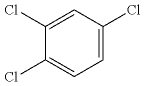 | H17 |
| 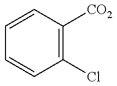 | H18 |
| 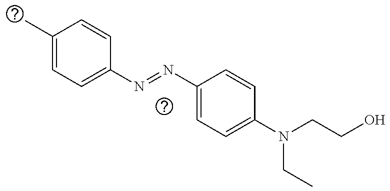 [SMILES: CCN(CCO)c1ccc(/N=N/c2ccc(C)cc2)cc1] | K05 |

Canonical Donors

| | |
|---|---|
| 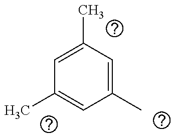 | X01 |
| 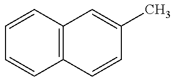 | X02 |
| 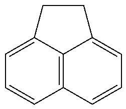 [SMILES: c1cc2c3c(cccc3c1)CC2] | X03 |
| 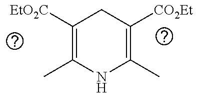 [SMILES: CCOC(=O)C1=C(C)NC(C)=C(C(=O)OCC)C1] | X04 |
| 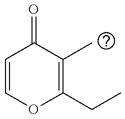 | X05 |
| 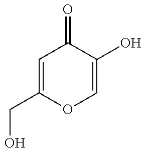 | X06 |
| 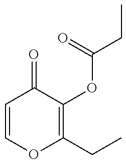 | X07 |
| 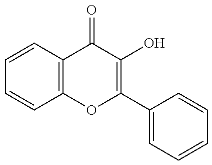 [SMILES: O=c1c(O)c(-c2ccccc2)oc2ccccc12] | X08 |

Indole, Carb [symbol] , and Hydrazine Functionality [including salts]

| | |
|---|---|
| 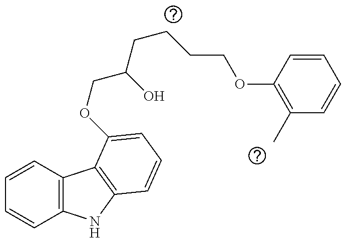 [SMILES: Cc1ccccc1OCCCCC(O)COc1cccc2[nH]c3ccccc3c12] | I01 |
| 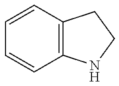 | I02 |
| 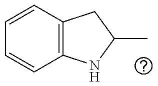 | I03 |
| 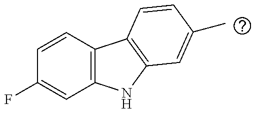 | I04 |
| 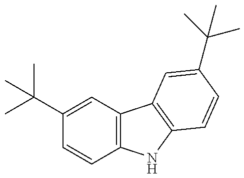 [SMILES: CC(C)(C)c1ccc2[nH]c3ccc(C(C)(C)C)cc3c2c1] | I05 |
| 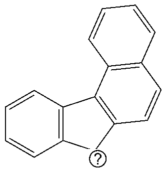 [SMILES: c1ccc2c(c1)Cc1ccc3ccccc3c1-2] | I06 |
| 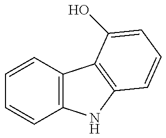 [SMILES: Oc1cccc2[nH]c3ccccc3c12] | I07 |
| 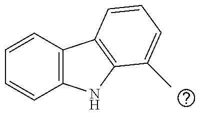 | I08 |
| 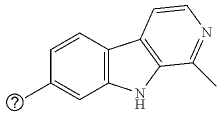 | G12 |
| 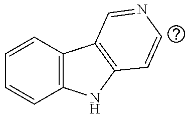 [SMILES: c1ccc2c(c1)[nH]c1ccncc12] | I09 |
| 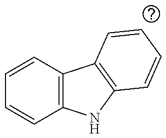 [SMILES: c1ccc2c(c1)[nH]c1ccccc12] | I10 |
| 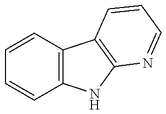 [SMILES: c1ccc2c(c1)[nH]c1ncccc12] | I11 |
| 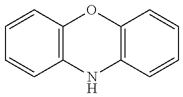 [SMILES: c1ccc2c(c1)Nc1ccccc1O2] | I12 |
| 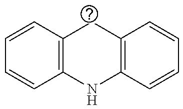 [SMILES: c1ccc2c(c1)Cc1ccccc1N2] | I13 |
| 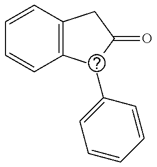 [SMILES: O=C1Cc2ccccc2C1c1ccccc1] | H31 |
| 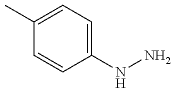 | J01 |
| 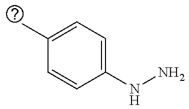 | J02 |
| 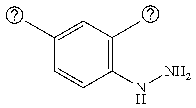 | J03 |
| 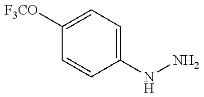 [SMILES: NNc1ccc(OC(F)(F)F)cc1] | J04 |
| 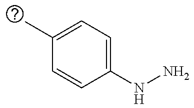 | J05 |
| 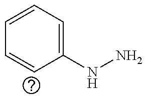 | J06 |
| 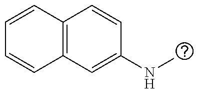 | J07 |
| 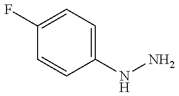 | J08 |
| 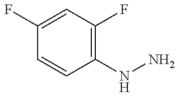 | J09 |
| 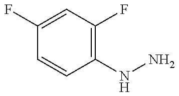 | J10 |
| 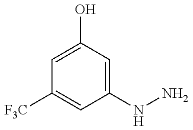 [SMILES: NNc1cc(O)cc(C(F)(F)F)c1] | J11 |
| 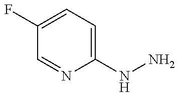 | J12 |
| 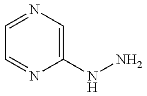 | J13 |
| 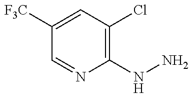 [SMILES: NNc1ncc(C(F)(F)F)cc1Cl] | J14 |

[symbol]

indicates data missing or illegible when filed

### A. Key Analytes of Interest:

Initial analytes will be mycotoxins, secondary metabolites from certain pathogenic fungi (e.g. Penicillium, Aspergillis, Fusarium, and Alternaria fungi) that are prevalent in cereal grains and thus can contaminate animal feed and human food. There are other substrates at risk of mycotoxin contamination as well as other analytes amenable to this diagnostic technique. Prevalent mycotoxins of interest are shown in the figure below, namely aflatoxin (AF; B₁, B₂, G₁, G₂, and M₁; only B₁ shown), zearalenone (ZEN), ochratoxin A (OTA), and deoxynivalenol (DON).

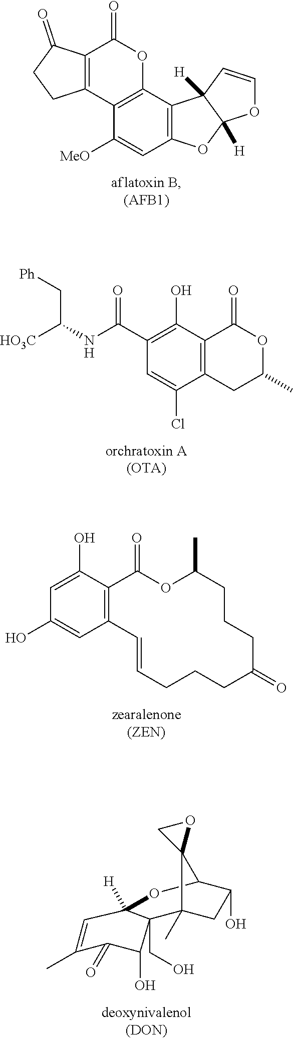

The first three toxins follow directly with the concepts and models above. Deoxynivalenol (DON) may follow directly with the concepts above but may also involve a variation of the strategy in which DON is pre-functionalized before partaking in any number of possible photochemical detection processes. Pre-functionalization may be utilized in the detection of any number of other analytes, including the others listed above and below. Other mycotoxins of interest that are prospective targets for this exciplex-based detection strategy are sterigmatocystin, cyclopiazonic acid, ergot alkaloids (ergotamine, ergovaline, ergocryptine), patulin, citrinin, moniliformin, citreoviridin, alternariol, alternariol monomethyl ether, paxilline, PR toxin, roquefortine C, tenuazonic acid, lolitrem B, penitrem A, fusaproliferin, mycophenolic acid, emodin, aurofusarin, gliotoxin, wortmannin.

In addition to the free toxins, certain mycotoxins are prone to biochemical transformations by the producing fungi, the plant, or the associated microbiome. This technology may be tailored to be sensitive or insensitive to these modifications, allowing for detection of the functionalized mycotoxins (aka. “masked mycotoxins”) in conjunction with or in addition to the free mycotoxins. Masked mycotoxins of note are: DON-3-glucoside, DON-3-acetate, DON-15-acetate, ZEN-14-sulfate.

### B. Buffer Components:

While the detector compounds are designed to directly manipulate the photophysical properties of the toxins, the buffer matrix enables the detection of those altered spectroscopic profiles. The buffer must provide a few generic functions in order to optimally support the diagnostic technique: 1) efficient recovery of mycotoxins in the filtrate; 2) precipitation, sequestration, and/or degradation of background contaminants (plant-derived or otherwise); 3) normalize the background noise across multiple feed types; 4) provide the molecular-level environment that promotes the non-covalent associations of interest; 5) provide the molecular-level environment that promotes quantum yield (both in terms of photon absorbance and emission).

### EXAMPLES

An ochratoxin A assay kit prototype generates quantitative data for OTA contamination in a variety of feeds and feed ingredients. Both naturally-contaminated and exogenously-contaminated samples are assessed to provide preliminary assessment of the versatility of the present diagnostic concept.

### Example Testing Protocol for Examples 1-12

The following tests for Examples 1-12 include making of samples and evaluating mycotoxin content:
Trials:
   1 or more calibration curves (as needed)
   unit test with sample of interest (naturally-contaminated or exogenously contaminated in the method provided)

### A. Prototype Kit Contents and Equipment Needed

### Provided:

Ochratoxin A stock solution in DMSO
Buffer, in liquid formulation
   Liquid formulations of buffers are prepared by dissolving listed buffering components and other additives in water (˜80% of final target volume) followed by adjustment of the pH using base or acid [potassium hydroxide and hydrochloric acid are representative examples]; co-solvent is added, mixed solution is diluted to final volume, mixed, and final adjustment to volume and/or pH are made if applicable; detector compound is added as a stock solution [1000× stock solutions in DMSO are representative; this may be performed immediately prior to testing depending on the application; detector compound may also be added at the time of initial dissolution]
   This method may also employ solid formulations, see operation note below; solid formulations may be prepared following any numbers of methods standard to the field
Mixing Vials and Filtration Setups [Whatman Grade 4 filters (7 mm), small funnel, test tubes]
96-well platereader with fluorimetry capabilities (Em scan or Ex/Em 390/440 filter)
variations employing a portable spectroscopic instrument are discussed below
Standard lab equipment: variable-volume pipets; analytical balance, test tube racks, etc.
96-well plates (recommended: flat-bottom, black; must be able to hold 0.2 mL/well)

### B. Calibration Curve Generation Protocol

Prepare OTA serial dilution:
   Prepare 4% DMSO Buffer stock solution: 960 mL Buffer+40 mL DMSO
   Fill 4 vials with 150 mL 4% DMSO Buffer
   Prepare 80 μM OTA Stock solution: 192 mL Buffer+8 mL 2.0 mM OTA DMSO Stock
   Make first serial dilution by transferring 50 mL 80 μM OTA Stock in a vial containing 150 mL 4% DMSO Buffer; mix well by vortexing
   Repeat process with remaining vials to generate 5 vials of descending concentration; the resultant stock solutions should have the following concentrations: 80 μM, 20 μM, 5.0 μM, 1.25 μM, 0.3125 μM
Prepare 6 Filtration Setups (see Prototype Kit Contents; fold each filter in quarters, open to reveal cone)
Fill 6 Mixing Vials with 1.0 g Feed [feed should be pre-ground, composite sample]
Add 3.96 mL Buffer to each Feed Vial; label each vial to correspond to an OTA Stock solution, dedicating 1 vial for the 4% DMSO Buffer blank
in the case employing solid formulation, the solid is added directly to the feed sample, followed by addition of water.
Add 40 μL of each OTA Stock Solution to its respective Buffer-Feed Vial
Vortex vial for 15 seconds
   longer mixing time are needed for certain samples (between 15 sec and 3 min)
alternative methods of mixing are also viable
Pour slurry into Filtration Setup
Upon collecting ˜1.5 mL Filtrate for each sample, remove filter [see note below]
Briefly vortex test tube to mix, then aliquot 150 μL Filtrate into each of 3 wells in the recommended 96-well plate (3 replicates of 150 μL)
Insert plate into platereader and read; Platereader settings:
   Excitation=390 nm [any filter between 380-395 nm should be acceptable]
   Emission=440 nm [any filter between 430-460 nm should be acceptable]
   Integration Time=20 ms [extend if sensitivity is low]
   Gain=50% of instrument max [decrease if detector overloaded; increase if sensitivity is low]
   Number of Measurements (Points)=≥5
   Slit widths=adjust as based on signal intensity
   Test kit is also compatible with aforementioned portable detection device; settings of any given spectroscopic instrument may require alteration to accommodate the specific capabilities of the instrument
Average triplicate data, and subtract blank from OTA containing data points
Plot Concentration vs. Emission Signal; the resultant linear trendline will be used for subsequent interpolation from contaminated samples
For best results, repeat 3× and average the slope of the calibration curve
Volumes and ratios can be scaled to accommodate larger sample sizes

| Concentration Conversion Table | |
|---|---|
| Stock | |
| Concentration | Assay Conc (pre-filtration) |

| Vial | (μM) | in nM | in ppb |
|---|---|---|---|
| Initial Stock | 80 | 800 | 1292 |
| Dilution #1 | 20 | 200 | 323 |
| Dilution #2 | 5.0 | 50 | 80.8 |
| Dilution #3 | 1.25 | 12.5 | 20.2 |
| Dilution #4 | 0.3125 | 3.125 | 5.0 |
| Blank | 0 | 0 | 0 |

Note:
For certain feeds, 4 mL may not be enough to generate 1.5 mL filtrate

if <1.5 mL but >600 mL of filtrate are obtained after 4 min, proceed to mixing and pipetting steps
if after 4 min, <600 mL of filtrate are present, the filter can be compressed to collect additional liquid; this then requires transfer to a tube and centrifugation; aliquot from the supernatant and proceed
to avoid the latter, it is recommended that a test sample be prepared for each feed (particularly silages); the values above are amenable to scaling, thus if a 50% increase in sample is needed, sample prep would employ 1.5 g feed, 5.94 mL buffer, and 60 μL of OTA stocks
for larger samples (e.g. 10 g samples), use a larger filter paper (e.g. 11 cm diameter) and halt collection of filtrate after a defined time period (e.g. 1.5 min)

### C. Ochratoxin A (OTA) Detection Protocol

Fill 3 Mixing Vials with 1.0 g Feed [feed should be pre-ground, composite sample; scale amounts if desired or required]
Add 4.0 mL Buffer to each vial
Vortex vial for 15 seconds
Pour slurry into Filtration Setup
Upon collecting ˜1.5 mL Filtrate for each sample (or after a defined time point for larger samples), remove filter
Briefly vortex test tube to mix, then aliquot 150 μL Filtrate into each of 3 wells in the recommended 96-well plate (3 replicates of 150 μL for each of 3 vials)
Insert plate into platereader and read [see platereader settings above or employ portable detection device or other instrument]
Average each set of replicates, subtract background [blank from calibration curve stage], and interpolate toxin concentration based on calibration curve
   D. Data from Ochratoxin A (OTA) Calibration Curve and Detection Protocol

The protocols listed above were applied to the detection of ochratoxin A in corn. The resulting calibration curve data is shown below.

| | |
|---|---|
| Toxin Conc | Signal Data |

| nM | ppb | ΔEm | SD | S/N |
|---|---|---|---|---|
| 0.0 | 0.0 | 13 | 0.8% | . |
| 3.7 | 6.0 | 138 | 0.6% | 1.3% |
| 11 | 18 | 110 | 0.8% | 1.0% |
| 33 | 54 | 409 | 0.8% | 3.8% |
| 100 | 162 | 1491 | 1.0% | 13.9% |
| 300 | 485 | 3880 | 0.5% | 36.3% |

Based on the method employing the calibration curve data shown above, selected unit tests were performed in corn; the data is shown below.

The buffer components and detector compound employed for the representative data set above are as follows: 500 mM R20, pH 8, 100 mM S41, 35 mM B10, 15 mM POI [avg. m.w.=6000] containing 10 μM E03.

Alternative use cases for exciplex-based detection strategy include disease detection via metabolomics analysis, such as analysis of volatile organic compounds [VOCs] for human health, including allergens and animal health, such as bovine respiratory disease and detection of phenol and benzothiazole as predictors of infection or lack thereof. Appropriate methods utilizing spectroscopy, although different sampling than with feed, may include sweat, breath, saliva, blood, urine, fecal or tissue analysis, or nasal swabs, which would be the mode for a bovine respiratory disease sampling.

In terms of crop protection, predictive metrics for disease onset may be analyzed by metabolites from fungal pathogens, as in cereal grains, where suspected pathogens may include Aspergillus, Fusarium, Penicillium, etc., the same toxins we are seeking to detect in animal feed. Predictive metrics for additional fungal pathogens in additional crops are also possible. For instance, in strawberries the pathogen could be Neopestalotiopsis and the indicative metabolites could be oxysporone, afritoxinone A, afritoxinone B. The pathogen Botrytis may be present in strawberries or other fruit crops, as indicated by its associated biomarkers. In peanuts, the pathogen may include Aspergillus as indicated by aflatoxin.

Regarding food safety, detection of metabolites of infectious diseases on vegetables, fruit, or meat, detection of bacterial metabolites from Listeria or E. Coli, viral or fungal metabolites, including mycotoxins, are all achievable through the present invention. Furthermore, bioaccumulated environmental contaminants in meat, fish, or alternative proteins are detectable and quantifiable, along with fungal metabolites, such as mycotoxins, and algal metabolites, such as domoic acid in shellfish and mollusks. One may also use the present methods for detection of environmental contaminants, such as metals or specifically mercury, in swordfish and other large marine animals. Beverage mycotoxin contaminants in beer, wine or spirits are spectrally detectable, as well as environmental toxins such as soil or water contaminants oncluding dioxins or 2,3,7,8-Tetrachlorodibenzo-p-dioxin (TCDD).

Moreover, water contaminants from the sea, lake, wastewater, or drinking water are capable of being tested for dioxins, pharmaceuticals, narcotics, minerals and/or metals, microbe-derived cyanobacterial toxins, algae metabolites that are predictors of algal blooms and domoic acid. Air samples are possible to be tested for contaminants including VOCs.

Further tests may prove fruitful for narcotics or other (formerly) controlled substances by in-field identification of designer drugs, methamphetamine derivatives (phenethylamines), opioid/opiate derivatives, LSD derivatives, fentanyl derivatives, cannibinoids and functional analogs (alkylindoles, cyclohexylphenols, indazole carboxamides), tryptamine derivatives, cocaine derivatives (tropanes), phenylcyclohexylamine derivatives (i.e. PCP or ketamine analogs). Preferred methods are predictably performed by testing saliva, sweat, breath, urine, blood, etc. or by direct residue analysis. Further, cannabis/hemp quality control may be achievable for THC and CBD quantification, or through detection of mycotoxin contaminants.

The extraction buffer composition technologies are broadly designed for the solubilization of analytes from mixtures. The principal analytes of interest are mycotoxins as they are small-molecule toxins produced by fungi. Mycotoxins that contaminate cereal grains and the resultant products are the primary class of analytes of interest, though this extraction buffer technology is also applicable to alternative classes, e.g. mycotoxins on fruit crops, on nuts, on seeds, on spices, or in the home.

The primary function of the solubilized analytes is to enable quantification methods. Solubilized mycotoxins are detected through a novel exciplex-based detection methodology. All buffer components are envisioned for the broader purpose of extraction of other analytes besides mycotoxin extraction as a stand-alone function, which can be paired with any number of downstream functions, manipulations, analyses, etc. Disclosed is the present invention of extraction buffer technology used in conjunction with immunoassay-based quantification techniques. These novel methods provide structural identity of the individual components, overall performance of the fully composed extraction buffers, an ability to reliably extract mycotoxins and other analytes from a wide variety of substances/matrices, as well as the ability to perform this extraction function while still allowing for accurate quantification by the methodologies below.

Immunoassay techniques play an essential role in the existing analyte and mycotoxin detection landscape. Lateral flow devices (LFDs) are a primary contributor, representing the most commonly used method of on-site analysis of grains and other food/feed raw materials. ELISA assays and flow cytometry-based immunoassays are also employed, predominantly for off-site usage in third party labs. Immunoaffinity columns (IACs) are also employed as a means of purifying food and feed matrices of all forms. Mycotoxin-enriched eluents are then analyzed by any number of quantitative methods. Qualitative variants of these assays also exist, such as qualitative LFD strips. There are additional methods for quantifying mycotoxins that do not employ immunoassays, for example HPLC-based methods or LC-MS/MS-based methods. These are also viable methods with which the extraction buffer technology can be paired. All of these methods and others listed herein are used for the analysis of other analytes, each of which represents an additional use case for this extraction buffer technology.

An example of a prospective immunoassay-linked use would be the implementation of an extraction buffer in conjunction with LFD technologies. In this example, the feed or grain sample would be mixed with the extraction buffer, as detailed below. All possible formulations are hereby determined, including the instance in which the full extraction buffer is generated only upon a mixing step wherein one or more buffer component(s) is added directly to the sample as a solid. A liquid component such as water is then added separately, thus forming the full extraction buffer upon sample mixing. The mixing may be accomplished by any means. After mixing, various following steps may include separation by gravity filtration, settling and decanting, mechanical filtration, centrifugation, or any other viable alternatives or additions to this protocol. Upon separation, the filtrate would be directly loaded onto the LFD strip, and analysis would follow standard LFD protocols. A dilution step and/or additional separation step may be necessary. The dilution step could be performed with the same buffer or a different buffer, may or may not contain components critical to LFD function, and can be performed at any stage in the protocol. The full protocol may be employed for the quantification of one or more analyte, such as mycotoxin, from a single extraction.

Yet another aspect of the present invention would be the implementation in conjunction with IAC technologies. In this instance, the extraction buffer would be employed to solubilize the analyte(s), as described above. Raw extraction fluid, either with or without a filtration or other separation step(s) and with or without dilution, would be loaded onto an immunoaffinity column for the specific toxin(s) of interest. Additional buffer of the same type or a secondary buffer would then be employed to rinse the column of residual byproducts, while the toxin(s) of interest remain bound to the column. An elution buffer or solvent would then elute the toxin(s) of interest in an enriched and purified form, allowing for quantification. Quantification can be achieved by any number of methods, most common of which are fluorimetry, HPLC-based methods, or LCMS-based methods. Any and all of these downstream methods (with or without any additional sample manipulation steps) are considered a viable pairing, as these operate independently of the function of the extraction buffer technology.

The above aspects and any related protocols are proposed to be viable for any number of possible feed or food matrices. This includes, but is not limited to, cereal grains, whether pre- or post-harvest, such as corn, wheat, barley, oat, millet, rye, sorghum, rice, or triticale, non-cereal grains such as amaranth or quinoa, milled grains and byproducts, such as wheat mids or bran, downstream grain products like corn distiller's grains [aka. DDGS] in wet or dry form, partially or fully formulated feed or food products, silages, haylages, or forages.

The buffer composition design strategy and the specific components are intended to be broadly applicable for analyte extraction needs and beyond. Alternative analytes may include, but are not limited to, heavy metals, inorganic substances, biological molecules, biological metabolites, environmental contaminants of all kinds, pharmaceuticals and their downstream byproducts, petrochemicals and related byproducts, products and byproducts of manufacturing industries, controlled substances and their metabolites. The substances or matrices from which these analytes are intended to be extracted may include, but are not limited to, whether in raw, purified, or partially-purified forms, water from any source, soil, plant material and downstream products, animal-derived materials and fluids such as flesh, bone, urine, blood, sweat, mucus of all forms, and feces. Collections may come from living or dead animals. Further, the present invention may find utility for other biological sources such as molds, algal blooms, fungi, and bacterial cultures, manufacturing byproducts, petrochemicals, air, gaseous waste or product streams, food and feed products, including the raw materials, intermediate formulations, and final products.

In accordance with the above-noted advantages and desires of the industry, the present invention provides new and superior extraction buffer technologies. These new technologies are usable beyond the previously disclosed support for exciplex based diagnostic technologies. The present disclosure includes new methods of solubilizing analytes from mixtures for generating wide spread spectroscopic detectable signals. By the addition of detector compounds and buffers to samples of contaminated animal and human feed, there is provided a further capability to detect human and animal diseases by detecting metabolites of said diseases.

The speed and simplicity of these improvements with the instant photochemical detection method provides a competitive advantage in the market. Delivering the versatility to move on-farm will directly enable proactive mycotoxin remediation strategies. Promoting informed implementation of remediation products not only increases feed additive sales for animal nutrition firms but also improves the productivity and profitability for livestock farmers. More importantly, the collective effect is a more efficient and productive food chain, providing a much-needed contribution to food security and sustainability.

Therefore, preferred methods for detecting contaminants in human foods and animal feeds, including terrestrial production animals, aquatic production animals, and companion animals, as well as raw or processed feed/food ingredients comprise solubilizing analytes from mixtures of feed samples with an extraction buffer and using exciplex-based methods of detecting, identifying, and quantifying the analytes solubilized by the extraction buffer to find contaminants. In such methods, the solubilized analytes include mycotoxins, as well as those selected from the group including aflatoxins, zearalenones, ochratoxin A and/or deoxynivalenol (DON), and combinations thereof.

The buffer components shall include small molecules comprised of an acidic component and/or a basic functionality including an amine with a pK_{A} of the acid or the conjugate acid within the range of 3-12.

This method further comprising quantitatively analyzing the extraction buffer eluent that has been enriched in analyte. In certain aspects, the method also further comprises qualitatively analyzing for contaminants by exciplex-based diagnostic methods, wherein the detection and quantification is the result of exciplex-based diagnostic methods. The method of the exciplex-based detection is based on fluorescence, phosphorescence or absorbance, among other detection methods.

Suitable detector compounds to be combined with the extraction buffer responsible for exciplex formation and signal production include canonical acceptors, including the following:

Canonical Acceptors

| | |
|---|---|
| 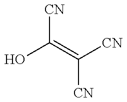 | A01 |
| 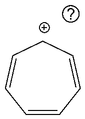 | A02 |
| 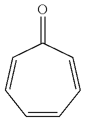 | A03 |
| 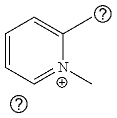 | A04 |
| 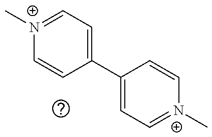 | A05 |
| 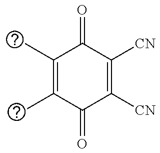 | A06 |
| 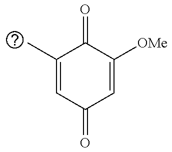 | A07 |
| 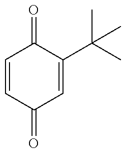 | A0 [symbol] |

[symbol]

indicates data missing or illegible when filed

Further suitable detection and quantification methods include performing with immunoassay techniques.

Although the invention will be described by way of examples herein, see below for specific aspects having certain features, it must also be realized that minor modifications that do not require undo experimentation on the part of the practitioner are covered within the scope and breadth of this invention. Additional advantages and other novel features of the present invention will be set forth in the description that follows and in particular will be apparent to those skilled in the art upon examination or may be learned within the practice of the invention. Therefore, the invention is capable of many other different aspects and its details are capable of modifications of various aspects which will be obvious to those of ordinary skill in the art all without departing from the spirit of the present invention. Accordingly, the rest of the description will be regarded as illustrative rather than restrictive.

### INDUSTRIAL APPLICABILITY

The present invention finds utility in the contaminant detection industry for human and animal feedstocks, in order to provide effective on-site testing methods.

What is claimed is:

1. A method for detecting contaminants in human foods and animal feeds, including terrestrial production animals, aquatic production animals, and companion animals, as well as raw or processed feed/food ingredients comprising:
solubilizing analytes from mixtures of feed samples with an extraction buffer;
detecting, identifying, and quantifying the analytes solubilized by the extraction buffer.

2. The method of claim 1, wherein the solubilized analytes include mycotoxins, and those selected from the group including aflatoxins, zearalenones, ochratoxin A and/or deoxynivalenol (DON), and combinations thereof.

3. The method of claim 1, wherein the buffer components include small molecules comprised of an acidic component and/or a basic functionality including an amine with a pK_{A} of the acid or the conjugate acid within the range of 3-12.

4. The method of claim 1, further comprising quantitatively analyzing the extraction buffer eluent enriched in analyte.

5. The method of claim 1, further comprising qualitatively analyzing for contaminants by exciplex-based diagnostic methods.

6. The method of claim 1, wherein the detection and quantification is the result of exciplex-based diagnostic methods.

7. The method of claim 6, wherein the exciplex-based detection is based on fluorescence, phosphorescence or absorbance.

8. The method of claim 7, wherein the detector compounds responsible for exciplex formation and signal production include anonical acceptors including

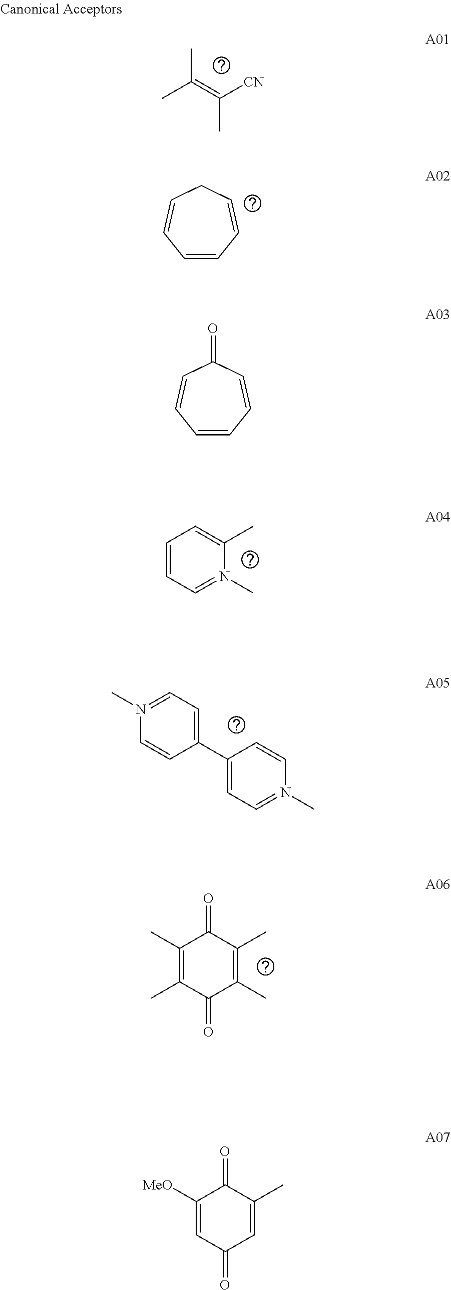

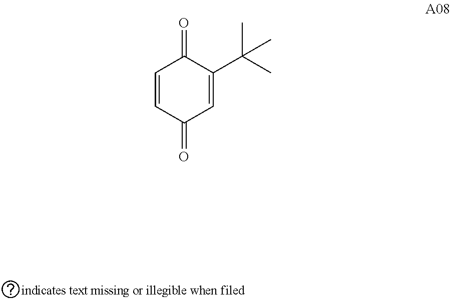

9. The method of claim 1, wherein the detection and quantification is performed with immunoassay techniques.
